(19) United States
(12) Patent Application Publication
(10) Pub. No.: US 2026/0335368 A1
(43) Pub. Date: Oct. 29, 2026

(54) MODULATION OF DELTA-LIKE NON-CANONICAL NOTCH LIGAND 1 IN STEM CELL DIFFERENTIATION AND FUNCTION AND USES THEREOF

(71) Applicant: Fred Hutchinson Cancer Center, Seattle, WA (US)

(72) Inventors: Irwin D. Bernstein, Seattle, WA (US); Suzanne Furuyama, Mountlake Terrace, WA (US); Kristen Johnson, Seattle, WA (US); Anna Koester, Seattle, WA (US); Brandon Hadland, Seattle, WA (US)

(73) Assignee: Fred Hutchinson Cancer Center, Seattle, WA (US)

(21) Appl. No.: 19/180,014

(22) Filed: Apr. 15, 2025

Related U.S. Application Data
(60) Provisional application No. 63/634,299, filed on Apr. 15, 2024.

(51) Int. Cl. A61K 35/28; C12N 5/074

(52) U.S. Cl. CPC A61K 35/28; C12N 5/0607; C12N 2501/125; C12N 2501/145; C12N 2501/2303; C12N 2501/2306; C12N 2501/26

(57) ABSTRACT

Methods of controlling stem cell proliferation and differentiation using a delta-like non-canonical Notch ligand 1 (DLK1) modulator is disclosed. Particularly, DLK1 maintains stem cell quiescence, delays stem cell differentiation, and enhances longer-term repopulation of stem cells. DLK1 knockdown shifts stem cells from quiescent to activated state. DLK1 modulators and DLK1 condensate-modifying therapeutics can be used to modulate DLK1 and its activity.

### CROSS-REFERENCE TO RELATED APPLICATIONS

This application claims priority to U.S. Provisional Patent Application No. 63/634,299 filed Apr. 15, 2024, which is incorporated herein by reference in its entirety as if fully set forth herein.

### STATEMENT REGARDING FEDERALLY SPONSORED RESEARCH OR DEVELOPMENT

This invention was made with government support under DK110563, and DK114777 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3EV7662.xml. The file is 58,311 bytes, was created on Apr. 15, 2025, and is being submitted electronically via Patent Center.

### FIELD OF THE DISCLOSURE

The current disclosure describes delta-like non-canonical Notch ligand 1 (DLK1) modulation as a means to control stem cell proliferation and differentiation. Particularly, DLK1 maintains stem cell quiescence, delays stem cell differentiation, and enhances longer-term repopulation of stem cells. DLK1 knockdown shifts stem cells from quiescent to activated state. In particular embodiments, stem cells include hematopoietic stem/progenitor cells (HSPC). In particular embodiments, stem cells include hematopoietic stem cells (HSC). DLK1 modulators and DLK1 condensate-modifying therapeutics can be used to modulate DLK1 and its activity.

### BACKGROUND OF THE DISCLOSURE

Primitive stem cells can differentiate to generate all cells of a given organism. Stem cells can undergo two types of cell division. Proliferative division gives rise to at least one identical daughter cell endowed with stem cell properties. Differentiative division, on the other hand, produces only progenitor cells with limited self-renewal potential or terminally differentiated postmitotic cells.

Somatic stem cells are undifferentiated cells that reside in differentiated tissues and have the properties of both self-renewal and generation of differentiated cell types. The differentiated cell types may include all or some of the specialized cells in the tissue. For example, hematopoietic stem cells give rise to all hematopoietic lineages, but do not give rise to stromal or other cells found in the bone marrow. Sources of somatic stem cells include bone marrow, blood, the cornea and the retina of the eye, brain, skeletal muscle, cartilage, bones, dental pulp, liver, skin, the lining of the gastrointestinal tract, and the pancreas. Stem cells in adults are usually quite sparse. Often they are difficult to identify, isolate, and purify. Often, somatic stem cells are quiescent until stimulated by the appropriate growth signals.

Stem cells are particularly important for renewal of tissues, underlying great hopes for regenerative medicine. Unfortunately, a major obstacle in adult stem cell research and their use in therapy, in contrast to stem cells of the developing embryo, is that they are difficult to expand both in vitro and in vivo. This is due to the different cell biology of somatic stem cells in comparison to stem cells of the developing embryo, and in particular to their quiescent state.

Quiescence is a reversible, non-proliferating state of cells. In a quiescent state, cells are not actively dividing but retain the ability to re-enter the cell cycle and proliferate when stimulated. A better understanding of how stem cell quiescence can be modulated is greatly need for research and therapeutic purposes.

### SUMMARY OF THE DISCLOSURE

The current disclosure describes delta-like non-canonical Notch ligand 1 (DLK1) modulation as a means to control stem cell proliferation and differentiation. Particularly, DLK1 maintains stem cell quiescence, delays stem cell differentiation, and enhances longer-term repopulation of stem cells. DLK1 knockdown shifts stem cells from a quiescent to an activated state. In particular embodiments, stem cells include hematopoietic stem/progenitor cells (HSPC). In particular embodiments, stem cells include hematopoietic stem cells (HSC). DLK1 modulators and DLK1 condensate-modifying therapeutics can be used to modulate DLK1 and its activity.

In particular embodiments, a DLK1 modulator includes a DLK1 agonist. In particular embodiments, a DLK1 agonist includes a protein, a small molecule, a nucleic acid, or a mimetic. In particular embodiments, a DLK1 agonist includes a DLK1 protein, a DLK1 extracellular domain, a sequence encoding DLK1 or its extracellular domain, or antisense oligonucleotides binding natural antisense oligonucleotides of DLK1 transcripts.

In particular embodiments, a DLK1 modulator includes a DLK1 antagonist. In particular embodiments, a DLK1 antagonist includes a protein (e.g., antibody), a small molecule, a nucleic acid, or a decoy. In particular embodiments, a DLK1 antagonist includes an anti-DLK1 antibody, an antisense oligonucleotide, or an RNA interference oligonucleotide (e.g., short interfering RNA).

In particular embodiments, a DLK1 condensate modifying therapeutic includes a substance, molecule, or compound that modulates TGFβ activity, gamma secretase complex cleavage, and/or integrin activity.

The present disclosure provides methods of delaying stem cell differentiation, shifting a population of stem cells from an activated state to a quiescent state, shifting a population of stem cells from a quiescent state to an activated state, enhancing DLK1 activity, minimizing DLK1 activity, and ameliorating the aging process in a subject.

### BRIEF DESCRIPTION OF THE SEVERAL VIEWS OF THE DRAWINGS

Many of the drawings submitted herein are better understood in color. Applicant considers the color versions of the drawings as part of the original submission and reserves the right to present color images of the drawings in later proceedings. The patent or application file contains at least one drawing executed in color. Copies of this patent or patent application publication with color drawing(s) will be provided by the Office upon request and payment of the necessary fee.

FIGS. 1A, 1B. Effect of delta-like non-canonical Notch ligand 1 (DLK1) knockdown or DLK1 overexpression on in vivo repopulating activity. Cord blood (CB) CD34+ cells were thawed (day 0) and allowed to recover overnight in Stemspan plus 5 growth factors (SS+5GF; stem cell factor (SCF), FMS-like tyrosine kinase 3 ligand (FLT3L), interleukin (IL)-6, thrombopoietin (TPO), all at 50 ng/ml, and IL-3, at 10 ng/ml). On day 1, cells were infected with lentivirus expressing an short hairpin RNA (shRNA) specific for Dlk1 or its scrambled control (1A) or DLK1-GFP or control GFP (1B). The sequence used for DKL1 knockdown was GGACGATGGCCTCTATGAATG (SEQ ID NO: 33). The scrambled control sequence was GGTTCGCCAATGCGTAAGAGT (SEQ ID NO: 34). On day 2, the expanded progeny of 50,000 cells were transplanted into each NOD scid gamma (NSG) mouse. (1A) For DLK1 knockdown, bone marrow aspirates (4- and 11-weeks post-transplant) or harvest (17-19 weeks post-transplant) were analyzed for total human engraftment (CD45%) of transduced (GFP⁺) cells. Each circle represents one mouse. (1B) For DLK1 overexpression, bone marrow was harvested from 3 independent experiments at 2-, 6- or 32-weeks post-transplant. CD34% of GFP⁺ human CD45 was determined for each harvest. P-values were determined using a t-test.

FIG. 2. Effect of DLK1 knockdown on hematopoietic stem/progenitor cell (HSPC) during in vitro endothelial cell (EC) co-culture. CB CD34⁺ cells were transduced with lentiviruses encoding DLK1 or SCR control shRNAs co-expressing GFP. Cells were cultured on ECs for 2 weeks in Stemspan+SCF, FLT3L, TPO, at 50 ng/mL. Immuno-phenotype of GFP⁺ cells was determined by flow cytometry. Left. Percent GFP⁺ CD34⁺ cells. Right. Percent GFP⁺ CD34^{neg} monocytic (CD14) and/or granulocytic (CD15) cells. P-value were determined using a t-test.

FIG. 3. Effect of DLK1 overexpression on HSPC growth in vitro. CB CD34⁺ cells were transduced with lentiviruses encoding DLK1-GFP or control GFP and cultured for 9 days in SS+5GF. Immuno-phenotype of GFP⁺ CD34⁺ CD90^{lo} cells was determined by flow cytometry. P-value was determined using a t-test. Each of seven independent experiments is represented by a different dot.

FIG. 4. Effect of extracellular domain (ECD) on in vivo repopulating activity. CB CD34+ cells were cultured in Stemspan plus 5 growth factors (SCF, FLT3L, IL-6, TPO, all at 50 ng/ml, and IL-3, at 10 ng/ml) in the presence of 10 ug/mL ECD (R&D) or vehicle control (PBS). On day 4, the expanded progeny of 10,000 cells were transplanted into each NSG mouse. Bone marrow aspirates performed at 3-4 weeks (short-term, left) or 10-15 weeks (longer-term, right) post-transplant were analyzed for total human engraftment (CD45%). Each circle represents one mouse. Black dots are from one experiment and grey dots are from another experiment. P-values were determined using a t-test. Y-axis is log scale.

FIGS. 5A-5H. Single cell analysis of DLK1 knockdown following cell-free culture. (5A) Representative Uniform Manifold Approximation and Projection (UMAP) and cluster analysis of combined DLK1 knockdown (KD) and Scrambled control (SCR) scRNAseq datasets from one experiment annotated by cell type based on canonical gene expression profiles. MPP=multipotent progenitors, MEP=Megakaryocyte/erythroid progenitors, GMP=granulocyte/monocyte progenitors. (5B) UMAP showing proliferation index (a transcriptional measure of cell cycle activity). (5C) Heatmap of gene set scores by cell type cluster for published gene sets associated with quiescent hematopoietic stem cells (HSC) state, activated, or cycling HSC/MPP state, proliferation index, or GMP. (5D) UMAP by sample. (5E) DLK1 expression by sample. (5F) Proportion of cells by cell type from SCR vs DLK1 KD samples. (5G) DLK1 transcript expression by cell type. (5H) Gene-set scores for genes associated with quiescent HSC state, activated HSC/MPP state, or immature myeloid bias (Velten, et al., Nat Cell Biol, 2017. 19(4): p. 271-281 (“Velten 2017”)) between cells from SCR vs DLK1 KD samples (limited to cells of qHSC and aHSC/MPP cell type clusters). * P<2E-13, Wilcoxon rank-sum test.

FIGS. 6A, 6B. EC co-culture expands the number of quiescent HSC. (6A) UMAP and cluster analysis of integrated one week and 2 week scRNAseq datasets with quiescent HSC (qHSC) annotation based on canonical gene expression profiles. Violin plots show expression of sex-specific genes XIST (expressed in females) and UTY (expressed in males) for two identified qHSC clusters. (6B) Bar graph showing total expanded qHSC numbers by EC co-culture time point.

FIG. 7. Effect of DLK1 knockdown on qHSC. Highly enriched CB derived HSC were transduced with lentiviruses encoding DLK1 or SCR control shRNAs co-expressing GFP and cultured on ECs for 1 week in Stemspan+SCF, FLT3L, TPO, all at 50 ng/ml. Dot plots shows distribution of GFP⁺ cells in G0, G1 and S/G2/M phases of the cell cycle (Eddaoudi, et al., Methods Mol Biol, 2018. 1686: p. 49-57; and Liang, et al., Cell Stem Cell, 2020. 26(3): p. 359-376 e7 (“Liang 2020”)).

FIGS. 8A, 8B. ECD inhibits TGFβ signaling and Notch Receptor Cleavage. (8A) KG1a-GFP cells were seeded in serum free media without (solid line) or with (dashed line) 10 μg/mL ECD. Cells were then treated without (black) or with (grey) 2 ng/ml TGFβ. After 1 h incubation, cells were fixed, permeabilized, stained with anti-pSMAD2/3 antibody, and analyzed with flow cytometry. Flow plots from one representative experiment are shown (left panel) and % pSMAD2/3 positive cells are shown from replicates over 4 independent experiments. P value for TGFβ vs TGFβ+ECD (right panel): <0.0001, determined using a student's t-test. (8B) Chinese hamster ovary (CHO) cells were seeded onto wells coated with 10 ug/mL of immobilized Delta^{extIgG} for 3 hrs in the presence of increasing concentrations of ECD (0, 1, 3 and 10 μg/mL) or the gamma secretase inhibitor DAPT at 10 ug/mL. Cells were harvested and analyzed with western blot using an antibody against Notch 1 receptor cleaved by gamma secretase at Valine 1744 (clone D3B8). Signal was normalized to actin per sample. Graph represents the average of 9 samples, analyzed in 3 independent experiments, error bars are standard error of mean (SEM).

FIGS. 9A, 9B. DLK1 subcellular localization. (9A) KG1a-DLK1-GFP cells were seeded on a poly-lysine coated cover glass then fixed and stained for ECD. Confocal images were acquired of the center of the cell using a Zeiss LSM 789. (9B) KG1a-DLK1-GFP cells were seeded on a poly-lysine and fibronectin coated cover glass then fixed and stained for ECD. Images were acquired of the surface plasma membrane (PM) of cells using stimulated emission depletion (STED) microscopy on a Lecia SP8.

FIG. 10. Full-length (FL) DLK1 and ECD exhibit phase separation (PS) tendencies. Cells were set up and imaged as described in FIG. 7. Ten images were analyzed per condition to determine the number of FL DLK1 condensates (left panel) and ECD condensates (right panel) per cell in control and 1,6-hexanediol (1,6-HD) treated samples. P values were determined using a two tailed students t test.

FIGS. 11A-11F. DLK1 co-localizes with TGFβR1 and PSEN1. (11A) KG1a-DLK1-GFP cells were seeded on a poly-lysine and fibronectin coated cover glass then fixed and stained with anti DLK1-ECD (594) and anti TGFβR1 (Atto-647N) antibodies. Images of the surface of the cell (PM) were acquired. Images were analyzed for ECD, GFP, and TGFβR1 condensates using FIJI. (11B) The area of each DLK1 condensate−/+ TGFβR1 shown in nm². (11C) ECD intensity per condensate−/+TGFβR1 is shown in arbitrary units (AU). (11D) Cells were prepared for microscopy as in FIG. 11A and stained with anti DLK1-ECD (594) and anti PSEN1 (Atto-647N) antibodies. (11E) The area of each DLK1 condensate−/+PSEN1 shown in nm². (11F) ECD intensity per condensate−/+PSEN1 is shown in AU. P values were determined using a two tailed students t-test. Scale bars are 5 μm.

FIGS. 12A, 12B. DLK1 alters receptor dynamics. (12A) KG1a cells ere pretreated with 0, 1, or 10 μg/mL ECD then stimulated with 2 ng/ml TGFβ for 1 h before fixing and staining for TGFβR1 and pSmad2/3 levels. Error bars are SEM. (12B) KG1a-DLK1-GFP cells were fixed and stained for PSEN1 (584) and either Rab7-647 (late endosomes) or Lamp1-647 (lysosome). Scale bars are 10 μm.

FIG. 13A illustrates a cartoon representation of ECD proteins produced and tested in this study. WT ECD protein (A), ECD mutant lacking the IDR (B) and ECD mutant lacking EGF1-EGF6 (C).

FIGS. 13B, 13C illustrate TGFB reporter assay in KG1a-GFP (13B) and KG1a-DLK1-GFP (13C) cells. The indicated KG1a cell line was treated without or with 10, 30, 100, 300, or 1000 nM ECD protein WT or mutant overnight then stimulated with TGFB ligand for 1 h. Cells were fixed, permeabilized, and stained for pSmad2/3. pSmad2/3 MFI was determined with flow cytometry and normalized to no ECD addition. Significance was determined with a 2way ANOVA with Dunnett's multiple comparisons test in Prism.

### DETAILED DESCRIPTION

The Notch ligand family member, Delta like homologue 1 (DLK1), is essential to the development and homeostasis of tissues within each of the three germ layers. While broadly utilized during embryogenesis, DLK1 is restricted in usage to distinct lineages within the adult. DLK1 is widely expressed during embryogenesis where it regulates stem cells to control organ size. In the adult, DLK1 expression is limited to select tissues, including hematopoietic stem cells (HSC) (Traustadottir et al., Cytokine Growth Factor Rev. 2019; 46:17-27), where it has been shown to enhance the generation of murine HSC ex vivo and to be involved in the maintenance of murine long-term repopulating HSC in vivo (Huang, et al., Exp Hematol Oncol, 2023. 12(1): p. 11; and Moore, et al., Proc Natl Acad Sci USA, 1997. 94(8): p. 4011-6). Yet, the precise effects of DLK1 on stem cell growth and differentiation remain elusive.

The DLK1 gene encodes a single pass transmembrane protein composed of an extracellular domain (ECD) with an N-terminal signal peptide and six epidermal growth factor (EGF)-like domains; a juxtamembrane region containing an ADAM17/TNFα converting enzyme (TACE)-dependent proteolytic cleavage site; and a short cytoplasmic tail. Upon its insertion into the plasma membrane, DLK1 can remain membrane-bound or be proteolytically cleaved by TACE to generate a soluble ECD fragment. DLK1 is also regulated at the level of pre-mRNA splicing; in humans, the two predominant spliced isoforms differ in their incorporation of the TACE cleavage site (Traustadottir et al., Cytokine Growth Factor Rev. 2019; 46:17-27). The usage of the membrane-bound versus proteolytically cleaved isoforms varies dependent on biological context. Importantly, the generation of a soluble DLK1 ECD fragment allows DLK1 to function in both an autocrine and paracrine manner, where it may amplify or suppress membrane-bound DLK1 function.

DLK1 is 383 amino acids in length. This sequence is available under UniProt accession number P80370, NIH gene ID 8788, and includes:

(SEQ ID NO: 1)
MTATEALLRVLLLLLAFGHSTYGAECFPACNPQNGFCEDDNVCRCQPGW
QGPLCDQCVTSPGCLHGLCGEPGQCICTDGWDGELCDRDVRACSSAPCA
NNRTCVSLDDGLYECSCAPGYSGKDCQKKDGPCVINGSPCQHGGTCVDD
EGRASHASCLCPPGFSGNFCEIVANSCTPNPCENDGVCTDIGGDFRCRC
PAGFIDKTCSRPVTNCASSPCQNGGTCLQHTQVSYECLCKPEFTGLTCV
KKRALSPQQVTRLPSGYGLAYRLTPGVHELPVQQPEHRILKVSMKELNK
KTPLLTEGQAICFTILGVLTSLVVLGTVGIVFLNKCETWVSNLRYNHML
RKKKNLLLQYNSGEDLAVNIIFPEKIDMTTFSKEAGDEEI.

The extracellular domain of DLK1 (ECD) spans from residue numbers 1-304 of the DLK1 sequence and includes:

(SEQ ID NO: 2)
MTATEALLRVLLLLLAFGHSTYGAECFPACNPQNGFCEDDNVCRCQPGWQ
GPLCDQCVTSPGCLHGLCGEPGQCICTDGWDGELCDRDVRACSSAPCANN
RTCVSLDDGLYECSCAPGYSGKDCQKKDGPCVINGSPCQHGGTCVDDEGR
ASHASCLCPPGFSGNFCEIVANSCTPNPCENDGVCTDIGGDFRCRCPAGF
IDKTCSRPVTNCASSPCQNGGTCLQHTQVSYECLCKPEFTGLTCVKKRAL
SPQQVTRLPSGYGLAYRLTPGVHELPVQQPEHRILKVSMKELNKKTPLLT
EGQA.

The extracellular domain of DLK1 ECD contains modular interacting domains (domains that interact with other proteins to modulate signaling), low complexity sequence domains (domains that are composed of similar amino acids resulting in low complexity within that domain) and a combination of ordered and intrinsically disordered regions that promote homo- and hetero-interactions to modulate cellular signaling.

The current disclosure describes delta-like non-canonical Notch ligand 1 (DLK1) modulation as a means to control stem cell proliferation and differentiation. Particularly, DLK1 maintains stem cell quiescence, delays stem cell differentiation, and enhances longer-term repopulation of stem cells. DLK1 knockdown shifts stem cells from quiescent to activated state. In particular embodiments, stem cells include hematopoietic stem/progenitor cells (HSPC). In particular embodiments, stem cells include hematopoietic stem cells (HSC). DLK1 modulators and DLK1 condensate-modifying therapeutics can be used to modulate DLK1 and its activity.

This disclosure provides novel insights into the design of DLK1 condensate-modifying therapeutics to expand quiescent HSC populations for stem cell regenerative medicine. Uses include the expansion of true repopulating HSC for transplantation, enhancing the effectiveness of HSC-based gene therapy as well as the prevention of HSC aging and its consequences. Since DLK1 is misexpressed in many aggressive, treatment resistant cancers, DLK1 modulators and therapeutics will also be directly applicable to the development of DLK1 inhibition strategies to reduce disease progression, including rendering cancer stem cells susceptible to conventional chemotherapy. In particular embodiments, DLK modulators include agents that enhance or reduce DLK1 activity and/or DLK1 expression.

In particular embodiments, methods disclosed herein include genetically modifying HSC and expanding genetically modified HSC ex vivo. In particular embodiments, genetically modified and expanded HSC could be returned to a quiescent, transplantable state after gene modification as part of a gene therapy approach using methods disclosed herein.

The present disclosure provides methods of delaying stem cell differentiation, shifting a population of stem cells from an activated state to a quiescent state, shifting a population of stem cells from a quiescent state to an activated state, enhancing DLK1 activity, minimizing DLK1 activity, and ameliorating the aging process in a subject.

For in vivo methods, it is known that during aging, HSC are reduced with an increase in more mature myeloid precursors. Thus, particular embodiments include using agents that enhance DLK1 activity to increase the robustness of stem cells to ameliorate the aging process.

In particular embodiments, DLK modulators include DLK1 inhibitors, DLK1 condensate-modifying therapeutics, DLK1 expression constructs, DLK1 ECD, DLK1 peptides, or antibodies.

In particular embodiments, a DLK1 modulator includes a DLK1 agonist. In particular embodiments, a DLK1 agonist includes a protein, a small molecule, a nucleic acid, or a mimetic. In particular embodiments, a DLK1 agonist includes a DLK1 protein, a DLK1 extracellular domain, a sequence encoding DLK1 or its extracellular domain, or antisense oligonucleotides that bind a natural antisense oligonucleotide that binds DLK1 transcripts.

In particular embodiments, a DLK1 modulator includes a DLK1 antagonist. In particular embodiments, a DLK1 antagonist includes a protein (e.g., antibody), a small molecule, a nucleic acid, or a decoy. In particular embodiments, a DLK1 antagonist includes an anti-DLK1 antibody, an antisense oligonucleotide, or an RNA interference oligonucleotide (e.g., short interfering RNA).

In particular embodiments, a DLK1 condensate modifying therapeutic includes a substance, molecule, or compound that modulates, TGFβ activity, gamma secretase complex cleavage, and/or integrin activity.

Aspects of the current disclosure are now described in more supporting detail as follows: (i) DLK1 Modulators; (i-a) Exemplary DLK1 Agonists; (i-b) Exemplary DLK1 Antagonists; (ii) DLK1 Condensate-Modifying Therapeutics; (iii) Genetic Engineering Techniques; (iv) Compositions for Administration; (v) Formulations; (vi) Methods of Use; (vii) Exemplary Embodiments; (viii) Experimental Examples; and (ix) Closing Paragraphs. These headings are provided for organizational purposes only and do not limit the scope or interpretation of the disclosure.

(i) DLK1 Modulators. The present disclosure provides DLK1 modulators to control stem cell proliferation and differentiation. As used herein, the term “modulator” refers to a composition that modulates one or more physiological or biochemical events. In particular embodiments, a DLK1 modulator inhibits one or more biological activities associated with DLK1. In particular embodiments, the modulator includes a protein, a small molecule, a nucleic acid, a mimetic, or a decoy. In particular embodiments, a DLK1 modulator causes modulation of or modulates DLK1 expression and/or DLK1 activity.

“Modulation of DLK1 expression and/or DLK1 activity”, as used herein, refers to an increase or decrease in DLK1 expression and/or DLK1 activity that can be a result of, for example, interaction of an agent with a polynucleotide or polypeptide, inhibition of transcription and/or translation (e.g., through antisense or siRNA interaction with a gene or transcript, through modulation of transcription factors that facilitate expression), and the like. For example, modulation of expression and/or activity refers to i) an increase in expression and/or activity or ii) a decrease in expression and/or activity. In particular embodiments, the agent can act on DLK1 directly or indirectly. For example, an agent can directly express DLK1 or directly inhibit DLK1. In other embodiments, an agent causes modulation of DLK1 expression and/or activity by acting on a related polynucleotide or polypeptide such as those involved in the TGFβ or Notch signaling pathway.

The terms “agonist” or “activator” refer to an agent that upregulates (e.g., activates or enhances) at least one bioactivity of a protein (e.g., expression or activity). An agonist may be a compound which increases the interaction between a protein and another molecule, e.g., a target peptide or enzyme substrate. An agonist may also be a compound that increases expression of a gene or which increases the amount of protein expressed.

The terms “antagonist” or “inhibitor” refer to an agent that downregulates (e.g., suppresses or inhibits) at least one bioactivity of a protein (e.g., expression or activity). An antagonist may be a compound which inhibits or decreases the interaction between a protein and another molecule, e.g., a target peptide or enzyme substrate. An antagonist may also be a compound that downregulates expression of a gene or which reduces the amount of protein expressed.

In particular embodiments, a DLK1 modulator that is a protein includes a DLK1 protein, a DLK1 peptide, an anti-DLK1 antibody, or a protein, peptide, or antibody that modulates DLK1 or the TGFβ or Notch signaling pathway.

A protein is a macromolecule composed of one or more long chains of amino acids that fold into specific three-dimensional structures, enabling them to perform a wide variety of biological functions. Proteins serve as enzymes, structural components, signaling molecules, and regulators in various cellular processes. In particular embodiments, a protein includes DLK1.

A peptide refers to an oligomer of at least two contiguous amino acids, linked together by a peptide bond. In particular embodiments, a peptide includes 2 to 40 amino acids. Exemplary peptides include any portion of a DLK1 protein such as the extracellular domain (ECD). In some embodiments, the peptide includes a domain of a larger protein. The term “domain”, when used in connection with a polypeptide, refers to a specific region within such polypeptide that comprises a particular structure or mediates a particular function. In the typical case, a domain of a polypeptide is a fragment of the polypeptide.

The term “antibody” refers to intact immunoglobulin molecules as well as to fragments thereof, such as Fab, Fab′, F(ab′)2, scFv, and Fv fragments, which are capable of binding an epitopic determinant. In particular embodiments, antibodies can have agonistic or inhibitory effects upon binding to their target antigen. An antibody can include monoclonal and polyclonal antibodies, single chain antibodies, chimeric antibodies, bifunctionally-specific antibodies, humanized antibodies, human antibodies, and complementary determining region (CDR)-grafted antibodies, that are specific for the target protein or fragments thereof.

In particular embodiments, the DLK1 modulator includes a small molecule. The term “small molecule” refers to a compound, which has a molecular weight of less than 5 kD, less than 2.5 kD, less than 1.5 kD, or less than 0.9 kD. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened for DLK1 modulating activity. In particular embodiments, the small molecule can increase or decrease DLK1 or TGFβ or Notch signaling.

In particular embodiments, the DLK1 modulator includes a nucleic acid. The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides. In particular embodiments, the nucleic acid can increase or decrease DLK1 or TGFβ or Notch signaling. In particular embodiments, nucleic acids include sequences encoding DLK1 or peptides or variants thereof. In particular embodiments, nucleic acids include sequences that modulate the TGFβ or Notch signaling pathways.

In particular embodiments, nucleic acids include oligonucleotides. As used herein, the term “oligonucleotide” refers to a series of linked nucleotide residues. Oligonucleotides include antisense and siRNA oligonucleotides. Oligonucleotides include portions of a nucleotide sequence and have at least 5, 7, or 10 nucleotides and as many as 500 nucleotides. In some embodiments oligonucleotides comprise from 5 nucleotides to 50 nucleotides, from 10 nucleotides to 30 nucleotides, and from 20 nucleotides to 25 nucleotides. Oligonucleotides may be chemically synthesized and can also be used as probes. In some embodiments oligonucleotides are single stranded. In some embodiments oligonucleotides include at least one portion which is double stranded.

In some embodiments the oligonucleotides are antisense oligonucleotides (ASO). As used herein, the term “antisense oligonucleotide” refers to an unmodified or modified nucleic acid having a nucleotide sequence complementary to a polynucleotide sequence (e.g., DLK1 polynucleotide sequence), where the antisense polynucleotide is capable of hybridizing to the polynucleotide sequence. ASOs can be used for downregulation or upregulation. In the cases wherein an ASO is used as a DLK1 agonist, it can upregulate gene expression by targeting specific RNA sequences to correct aberrant splicing or increase translation efficiency. In particular embodiments, ASOs can include antisense RNA or antisense DNA.

In some embodiments the oligonucleotides are RNA interference oligonucleotides (RNAi oligonucleotides). The terms “RNAi oligonucleotides”, “siRNA oligonucleotides”, “short interfering RNA”, or “siRNA” are used interchangeably and refer to oligonucleotides that work through post-transcriptional gene silencing, also known as RNA interference (RNAi). The terms refer to molecules capable of RNA interference “RNAi”, (see Kreutzer et al., WO 00/44895; Zernicka-Goetz at al. WO 01/36646; Fire, WO 99/32619; Mello and Fire, WO 01/29058). siRNA molecules are generally RNA molecules but further encompass chemically modified nucleotides and non-nucleotides. siRNA gene-targeting experiments have been carried out by transient snRNA transfer into cells (achieved by such classic methods as liposome-mediated transfection, electroporation, or microinjection). Molecules of siRNA are 21- to 23-nucleotide RNAs, with characteristic 2- to 3-nucleotide 3′-overhanging ends resembling the RNase III processing products of long double-stranded RNAs (dsRNAs) that normally initiate RNAi.

Additional nucleic acids include short interfering RNA (siRNA); micro interfering RNA (miRNA); a small, temporal RNA (stRNA); or a short, hairpin RNA (shRNA); small RNA-induced gene activation (RNAa); small activating RNA (saRNA); or guide RNA (gRNA).

In particular embodiments, the DLK1 modulator includes a mimetic. The term “mimetic” refers to biologically active compounds that mimic the biological activity of a peptide or a protein. Here, the term mimetic is used to describe a substance, molecule, or compound that imitates the behavior of another entity (e.g., DLK1).

In particular embodiments, the DLK1 modulator includes a decoy. A decoy is a molecule that mimics the structure or function of a native ligand, receptor, or signaling component, thereby competitively inhibiting or altering its biological activity. Decoys can be used to modulate cellular pathways by sequestering ligands, blocking receptor interactions, or interfering with downstream signaling events.

DLK1 modulators include variants, alleles, homologs, mutants, derivatives, fragments, and complementary sequences thereto. In many cases, the DLK1 modulators act on DLK1 or molecules in the TGFβ or Notch signaling pathways. Targets of DLK1 modulators can also include family members, variants, mutants, single-nucleotide polymorphisms (SNPs), noncoding sequences, alleles, species variants, fragments and the like.

In particular embodiments, DLK1 modulators can be found in PCT Publication No. WO 2012/052594, WO 2010/107740, U.S. Pat. No. 9,708,604, U.S. patent application No. 20250042992, U.S. patent application No. 20240409642, and U.S. Publication No. 20240092857.

(i-a) Exemplary DLK1 Agonists. In particular embodiments, a DLK1 modulator increases DLK1 expression and/or DLK1 activity. DLK1 modulators that increase DLK1 expression and/or DLK1 activity include DLK1 agonists. DLK1 agonists can include proteins, small molecules, nucleic acids, or mimetics.

In particular embodiments, a DLK1 agonist includes a DLK1 protein, an extracellular domain of DLK1, an anti-DLK1 antibody, or sequences encoding a DLK1 protein, an extracellular domain of DLK1, an anti-DLK1 antibody.

In particular embodiments, the DLK1 agonist includes DLK1 proteins, sequences encoding DLK1 proteins, or variants thereof. In particular embodiments, the DLK1 protein includes the sequence of SEQ ID NO: 1. In particular embodiments, a sequence encoding DLK1 [Homo sapiens] (NCBI Reference

(SEQ ID NO: 35)
GGCAGCTCCCCGGCAGCGGCGGTGGAGAGCGCAGCGCGCAGCCCGGTGCAGCCCTGGC
TTTCCCCTCGCTGCGCGCCCGCGCCCCCTTTCGCGTCCGCAACCAGAAGCCCAGTGCGG
CGCCAGGAGCCGGACCCGCGCCCGCACCGCTCCCGGGACCGCGACCCCGGCCGCCCAG
AGATGACCGCGACCGAAGCCCTCCTGCGCGTCCTCTTGCTCCTGCTGGCTTTCGGCCACA
GCACCTATGGGGCTGAATGCTTCCCGGCCTGCAACCCCCAAAATGGATTCTGCGAGGATG
ACAATGTTTGCAGGTGCCAGCCTGGCTGGCAGGGTCCCCTTTGTGACCAGTGCGTGACCT
CTCCCGGCTGCCTTCACGGACTCTGTGGAGAACCCGGGCAGTGCATTTGCACCGACGGCT
GGGACGGGGAGCTCTGTGATAGAGATGTTCGGGCCTGCTCCTCGGCCCCCTGTGCCAAC
AACAGGACCTGCGTGAGCCTGGACGATGGCCTCTATGAATGCTCCTGTGCCCCCGGGTAC
TCGGGAAAGGACTGCCAGAAAAAGGACGGGCCCTGTGTGATCAACGGCTCCCCCTGCCA
GCACGGAGGCACCTGCGTGGATGATGAGGGCCGGGCCTCCCATGCCTCCTGCCTGTGCC
CCCCTGGCTTCTCAGGCAATTTCTGCGAGATCGTGGCCAACAGCTGCACCCCCAACCCAT
GCGAGAACGACGGCGTCTGCACTGACATTGGGGGCGACTTCCGCTGCCGGTGCCCAGCC
GGCTTCATCGACAAGACCTGCAGCCGCCCGGTGACCAACTGCGCCAGCAGCCCGTGCCA
GAACGGGGGCACCTGCCTGCAGCACACCCAGGTGAGCTACGAGTGTCTGTGCAAGCCCG
AGTTCACAGGTCTCACCTGTGTCAAGAAGCGCGCGCTGAGCCCCCAGCAGGTCACCCGTC
TGCCCAGCGGCTATGGGCTGGCCTACCGCCTGACCCCTGGGGTGCACGAGCTGCCGGTG
CAGCAGCCGGAGCACCGCATCCTGAAGGTGTCCATGAAAGAGCTCAACAAGAAAACCCCT
CTCCTCACCGAGGGCCAGGCCATCTGCTTCACCATCCTGGGCGTGCTCACCAGCCTGGTG
GTGCTGGGCACTGTGGGTATCGTCTTCCTCAACAAGTGCGAGACCTGGGTGTCCAACCTG
CGCTACAACCACATGCTGCGGAAGAAGAAGAACCTGCTGCTTCAGTACAACAGCGGGGAG
GACCTGGCCGTCAACATCATCTTCCCCGAGAAGATCGACATGACCACCTTCAGCAAGGAG
GCCGGCGACGAGGAGATCTAAGCAGCGTTCCCACAGCCCCCTCTAGATTCTTGGAGTTCC
GCAGAGCTTACTATACGCGGTCTGTCCTAATCTTTGTGGTGTTCGCTATCTCTTGTGTCAAA
TCTGGTGAACGCTACGCTTACATATATTGTCTTTGTGCTGCTGTGTGACAAACGCAATGCAA
AAACAATCCTCTTTCTCTCTCTTAATGCATGATACAGAATAATAATAAGAATTTCATCTTTAAA
TGAGTAAGAGAAATAAGTATGTTATTCTAAAATCTAAACTCAAATGAAATTTCAAAAAAGACC
AAAAAAAAACAAGGCAACAGAACCAGGGCTCAGTGCCGACGCCCCTACCCTGGGGGTCTC
GGCCACATGGTCCTCGTGAAACCGTTACGAGTGCTGTACATGACCACCCACTGTGCAAAG
AGCTACGATTGCTTTCGTTCGTTAATTCTCACACACCACATCCGACTCGCACTCACATCCAG
CTCAACATCGCTACCTTTAGATCTTTCCGATTGATTTGAGATTTAGCGGAGTGCAGCGGCT
GTGTGTCAGTCAGAGAGAGCCCCAGGTTGGCTGCTGGGGGGCCGGGCCTCTGTGCCAGC
CCTGCCACTAACTCGCTGTGTGATCCTTGGCGAGTCCCTATCCCAATCCTGGGCCCACTTT
CTCGCCCCTCTCGAGGGAGGGGTTTGAACACCGACCACCATGGTCCTCTCTGGCTCTAAA
ACTCTGAATTAGGAACGAGGGGGTATGAACCAAAACACTTCCTGACCCCAACAATTGGGAT
CTGATGAAGACGAATTCTCATCCTGGGATCCGAAAGGTTTTGGCCTATCCTGGATGAACGC
TTGATGTTGAAATTTATCCTAACTGTGTTTCTCAAAGTGGGGAGCCTCCTGGCCCACTTCCC
TTTTGGGAAAAAATAGCCCCTTTGAGTCATAATGTGCCATCTGGAGTTGCCACTTAGGCAG
CCAGCCCTCCCCCACCCCTCTTTGCCCACCCAGAACCCCAACACTATACCTGCCTCTACCC
TATAGAGACATCCCTTGAGACTTGAATGTACTTTGAGCCGCAAGTGCCCCTGGCAGAATCT
GAGCAGCAACACAGCCCTCACCAAGGTTTTCACAGGGATTAGTGGAAATTGTCACTTGTGG
GGCGCTTACTAGTTTTGTGGCAAATGTGGTCCAAGACCTGGCCCACACAGCCATGGGTTTA
CATTTGTGTTGCTGAGTTAATAATCCTCAGATCTCAAAAGACCTAGCCAGCTTCTGAATTTT
GAATTTGACTTTTTTTTTTTTTTTAACAAAGAGCATCTATCTTTTTTGCAAAAATAGAAAGAAA
TGGGACAGAGCTCTGTTTAGCTTTTCAGCTTTTGGGGTTGGACAGCTAAACAGCCCTTGGG
TACCAGATAGGAATATTTTGCTTCTTAGATTATTTGGATAAGAATCTATACTCTTAGCTTTAT
TGAGCATTGCCTGCCGTGTTTACCTTGCTCCAGGTGTTTATTTCTAAGTCATCATCACCTTC
GTCATCTACAGTCAGTCACATCAGTCACCCACACACAACTCACAGTCACCCATCCATTGGT
CACCTGCGCCATCGTCATGACACAATATCGACCTCATCACCTCATCGTCCTCCAGCCACTA
CCCAAGCCCCCTCATCCTGACTCAAAGCCCGACCAGCATCTGCCCAAACTCCCAACACCC
CACTCTCCCCACCAAGCCATTGGTCTCCTCACTCCCGGTCCTTTGTCATCCTGATTCCAAG
CCCCGCCCCACCCTCACCGCCTCCTCCGCATCATCTGCACTCTGCAGAGCCCCTCCTACC
CGCCTCTGTCCCCACCAGGCCCCTCCCTACCGTCAGCCCTGAGTCCCACAGCACGCGTCC
CCAGCCCCTCCCAGCCTCACCCCAAAAACCCTGTCACCCCACCGACCACCATCATCACTG
GCAAGCCCCTTCCCCTTCTCCAGCTCCCGTCCCCACTACCCCTCCCGTCCCCCAATCCTTC
CCCTCCTGTTAGCCCACCCCTCCTTTCCTATCACCCCCACCTTTCCCTTCCCATTCTCCCCA
CCCTTCCCCTCCGATCCCACCACCCTTCCCTTCCGATCCCCGTCATCCTTCCCCTCTGATC
CCCCACCCTTCCCCTCCTGTCCTCCCCAGCCCTCCCTTCCTATCACCCTCACCCTTCCCCT
CCCGTTCTCCCCACCCTTCCCCTCCTGTCTCCACCAGCCCTCCCTTCCTATCACCCCCACC
CTTCTCCTCCCTTCCCTCACCCCAAACCCCTGGGTCCTCCACCCCAATCTCTGACAACCGC
CCCAAGTCCCTCTTCCTTCCCCACGAAGTCATTCATAAGTCACCATCTCATCTCATTTTGAG
TTTGTTTTAAGAAATAAAAAAATTAAAAAGACAAAAAAAAGAATTGTTGGCATGTGTCAGGC
GGGCGTCTGGTACCCTGCCTGGCCTCTCTCAGCGTGAGACCTGGACTGAGCCTGCTAACG
ATTCTGTTCCTCAGTTTCCCCAAATAGAAAATTCTAAGATTGGTCAGATGATCAAATTATCTA
TGTTTTCGATCTTGATGCTATAGAGAACAGCCATGTTCCCCTGATAACAAGAAAAAGACGTC
CCACGGTCGAGGTATCCTTGACCAACTCCCCATCCATTCATTTGTTCACCGTCCACAACCC
ATCTGATCAGGAAGCATTTCCAGGCACCTGCGGAGCACTAGGCAGTGTGCGGAAGCTGCA
GGGCAGGCAGGACGAGGGGGCAGTCTGGAGGGGGGTGGACACACAGCTGTCCCCAGGG
AGCCCAGCGCGGTGCCGGTCACATGCCGTGGGAGTGGAGGGTCTTGAGCAGACGCCTCC
CTTGGAGAAGAGCCACCATCTCACCTTCCCACGCAACAAATTATCTATTACCAGCACCCGA
TGAGGCCGTCCCTCCCAAATTCCACAGTGCCCACCACTCGCACTACCCCCCCCGTGCCAC
AGAAGCAGTGTCAGCAGCCTCGCCACGGTCTTCACGTCATGCAGAAATGGTCATTCCATGT
TCTATGGCCACCAGATTCTGCTGAGAGTGTCGGGATCCTTGACTGCCCAGCCACACCTTGT
GTGTGTCTGGTGTAGTGTGTAGTTTACGAAACAAACGGAAAAGCTCAAAGACAGCGATCCA
AGACATGATCAGCAATTTGGTCCCAAATTAGGAGAGGAGGAAGAGGAGTATCAATTTTGTC
ACCAAAATGTTAAAAAAAATGTCCTTGGCGCTCTTTTTAGCTTTCCCGTTTACTACCAGTCC
AGACATGCAAAAAAACCTTGTTTATTGAAAAATAAACAACAAGGGCTAGA.

In particular embodiments, the extracellular domain of DLK1 includes the sequence of SEQ ID NO: 2. In particular embodiments, an agonist of DLK1 includes functional proteins originated from post-transcriptional modification of the sequence of SEQ ID NO: 2. In particular embodiments, DLK1 agonists include variants produced by cutting and splicing alternatives such as delta-like 1 homolog isoform CRA b [Homo sapiens] (GenBank: EAW81713.1), secredeltin (GenBank: AAY40461.1), brevideltinin [Homo sapiens] (GenBank: AAZ38943.1), or truncated brevideltinin [Homo sapiens] (GenBank: AAZ66768.1), or sequences encoding those variants.

In particular embodiments, delta-like 1 homolog isoform CRA b includes the sequence:

(SEQ ID NO: 36)
MTATEALLRVLLLLLAFGHSTYGAECFPACNPQNGFCEDDNVCRCQPGWQ
GPLCDQCVTSPGCLHGLCGEPGQCICTDGWDGELCDRDVRACSSAPCANN
GTCVSLDDGLYECSCAPGYSGKDCQKKDGPCVINGSPCQHGGTCVDDEGR
ASHASCLCPPGFSGNFCEIVANSCTPNPCENDGVCTDIGGDFRCRCPAGF
IDKTCSRPVTNCASSPCQNGGTCLQHTQGQAICFTILGVLTSLVVLGTVG
IVFLNKCETWVSNLRYNHMLRKKKNLLLQYNSGEDLAVNIIFPEKIDMTT
FSKEAGDEEI.

In particular embodiments, secredeltin includes the sequence:

(SEQ ID NO: 37)
MTATEALLRVLLLLLAFGHSTYGAECFPACNPQNGFCEDDNVCRCQPGWQ
GPLCDQCVTSPGCLHGLCGEPGQCICTDGWDGELCDRDVRACSSAPCANN
GTCVSLDDGLYECSCAPGYSGKDCQKKDGPCVINGSPCQHGGTCVDDEGR
ASHASCLCPPGFSGNFCEIVANSCTPNPCENDGVCTDIGGDFRCRCPAGF
IDKTCSRPVQQPEHRILKVSMKELNKKTPLLTEGQAICFTILGVLTSLVV
LGTVGIVFLNKCETWVSNLRYNHMLRKKKNLLLQYSSGEDLAVNIIFPEK
IDMTTFSKEAGDEEI.

In particular embodiments, brevideltinin includes the sequence:

(SEQ ID NO: 38)
MTATEALLRVLLLLLAFGHSTYGAECFPACNPQNGFCEDDNVCRCQPGWQ
GPLCDQCVTSPGCLHGLCGEPGQCICTDGWDGELCDRDVRACSSAPCANN
GTCVSLDDGLYECSCAPGYSGKDCQKKDGPCVINGSPCQHGGTCVDDEGR
ASHASCLCPPGFSGNFCEIVANSCTPNPCENDGVCTDIGGDFRCRCPAGF
IDKTCSSRSTAS.

In particular embodiments, truncated brevideltinin includes the sequence:

(SEQ ID NO: 39)
MTATEALLRVLLLLLAFGHSTYGAECFPACNPQNGFCEDDNVCRCQPGWQ
GPLCDQCVTSPGCLHGLCGEPGQCICTDGWDGELCDRDVRACSSAPCANN
GTCVSLDDGLYECSCAPGYSGKDCQKKDGPCVINGSPCQHGGTCVDDEGR
ASHASCLCPPGFSGNFCEIVANSCTPNPCENDGVCTDIGGDFRCRCSSRS
TAS.

In particular embodiments, a DLK1 agonist includes an ASO. In particular embodiments, a DLK1 agonist includes an ASO of 12 to 30 nucleotides in length that targets a region of a natural ASO of DLK1. In particular embodiments, the natural ASO of DLK1 includes the sequence:

(SEQ ID NO: 40)
GATGAAATTCTTATTATTATTCTGTATCATGCATTAAGAGAGAGAAAGAGGATTGTTTTTGCA
TTGCGTTTGTCACACAGCAGCACAAAGACAATATATGTAAGCGTAGCGTTCACCAGATTTG
ACACAAGAGATAGCGAACACCACAAAGATTAGGACAGACCGCGTATAGTAAGCTCTGCGG
AACTCCAAGAATCTAGAGGGGGCTGTGGGAACGCTGCTTAGATCTCCTCGTCGCCGGCCT
CCTTGCTGAAGGTGGTCATGTCGATCTTCTCGGGGAAGATGATGTTGACGGCCAGGTCCT
CCCCGCTGTTGTACTGAAGCAGCAGGTTCTTCTTCTTCCGCAGCATGTGGTTGTAGCGCAG
GTTGGACACCCAGGTCTCGCACTTGTTGAGGAAGACGATACCCACAGTGCCCAGCACCAC
CAGGCTGGTGAGCACGCCCAGGATGGTGAAGCAGATGGCCTGGCCCTCGGTGAGGAGAG
GGGTTTTCTTGTTGAGCTCTTTCATGGACACCTTCAGGATGCGGTGCTCCGGCTGCTGCAC
CGGCAGCTCGTGCACCCCAGGGGTCAGGCGGTAGGCCAGCCCATAGCCGCTGGGCAGA
CGGGTGACCTGCTGGGGGCTCAGCGCGCGCTTCTTGACACAGGTGAGACCTGTGAACTC
GGGCTTGCACAGACACTCGTAGCTCACCTGGGTGTGCTGCAGGCAGGTGCCCCCGTTCT
GGCACGGGCTGCTGGCGCAGTTGGTCACCGGGCGGCTGCAGGTCTTGTCGATGAAGCCG
GCTGGGCACCGGCAGCGGAAGTCGCCCCCAATGTCAGTGCAGACGCCGTCGTTCTCGCA
TGGGTTGGGGGTGCAGCTGTTGGCCACGATCTCGCAGAAATTGCCTGAGAAGCCAGGGG
GGCACAGGCAGGAGGCATGGGAGGCCCGGCCCTCATCATCCACGCAGGTGCCTCCGTGC
TGGCCCGTTGATCACACAGGGCCCGTCCTTTTTCTGGCAGTCCTTTCCCGAGTACCCGGG
GGCACAGGAGCATTCATAGAGGCCATCGTCCAGGCTCACGCAGGTCCTGTTGTTGGCACA
GGGGGCCGAGGAGCAGGCCCGAACATCTCTATCACAGAGCTCCCCGTCCCAGCCGTCGG
TGCAAATGCACTGCCCGGGTTCTCCACAGAGTCCGTGAAGGCAGCCGGGAGAGGTCACG
CACTGGTCACAAAGGGGACCCTGCCAGCCAGGCTGGCACCTGCAAACATTGTCATCCTCG
CAGAATCCATTTTGGGGGTTGCAGGCCGGGAAGCATTCAGCCCCATAGGTGCTGTGGCCG
AAAGCCAGCAGGAGCAAGAGGACGCGCAGGAG.

In particular embodiments, the antisense oligonucleotide that targets the natural antisense oligonucleotide of DLK1 includes the sequence:

(SEQ ID NO: 41)
GCCUCCUUGCUGAAGGUGGUCAUGUCG
and/or
(SEQ ID NO: 42)
CUGUGGGAACGCUGCUUAGAUCUCCUC.

In particular embodiments, ASOs include sequences set forth in SEQ ID NO: 41 or SEQ ID NO: 42. These oligonucleotides can include one or more modified nucleotides, shorter or longer fragments, modified bonds, and the like. In particular embodiments, oligonucleotides include sequences of at least 5 consecutive nucleotides of SEQ ID NO: 41 or SEQ ID NO: 42 and modulate expression and/or activity of DLK1.

Antibodies targeting (e.g., binding) any single or combination of the modular interacting domains of DLK1-ECD to modulate ECD function can also be used as DLK1 agonists.

(i-b) Exemplary DLK1 Antagonists. In particular embodiments, a DLK1 modulator decreases DLK1 expression and/or DLK1 activity. DLK1 modulators that decrease DLK1 expression and/or DLK1 activity include DLK1 antagonists or DLK1 inhibitors. DLK1 antagonists can include proteins, peptides, small molecules, nucleic acids, or decoys.

In particular embodiments, the DLK1 antagonist includes proteins or peptides that inhibit DLK1 activity (e.g., anti-DLK1 antibody), small molecules that inhibit DLK1, nucleic acids that inhibit DLK1, nucleic acids that knockout or knockdown DLK1, or DLK1 decoys. In particular embodiments, the DLK1 antagonist includes oligonucleotides. In particular embodiments, the DLK1 antagonist includes siRNA.

In particular embodiments, the DLK1 antagonist includes an antibody. In particular embodiments, the antibody includes an anti-DLK1 antibody with inhibitory properties. In particular embodiments, the anti-DLK1 antibody includes a variable heavy chain including the sequence: EVOLVESGGGLVQPGGSLRLSCAASGFSISDYYMAWVRQAPGKGLEWVANINYDGTNTYYAD SVKGRFTISRDNSKNTLYLQMNSLRAEDTA VYYCVRSYYYYGMEYWGQGTTVTVSS (SEQ ID NO: 43) and a variable light chain variable including the sequence:

(SEQ ID NO: 44)
DIQMTQSPSSLSASVGDRVTITCRASHDVSTAVAWYQQKPGKAPKLLIYS
ASYRYTGVPSRFSGSGSGTDFTLTISSLQPEDFATYYCQQHYRIPLTFGQ
GTKLEIK.

In particular embodiments, the anti-DLK1 antibody includes a variable heavy chain including the sequence: QVQLQESGPGLVKPSETLSLTCTVSGFSLSIYSVHWVRQPPGKGLEWIGLIWGGGSTDYNPSL KSRVTISKDTSKNQVSLKLSSVTAADTAVYYC ARKEGNYLWFAYWGQGTLVTVSS (SEQ ID NO: 45) and a variable light chain variable including the sequence:

(SEQ ID NO: 46)
DIVMTQSPDSLAVSLGERVTMNCKSSQSLLQSSNQKNYLAWYQQKPGQPP
KLLVYFASTRESGVPDRFSGSGSGTDFTLTISSVQAEDVAVYYCQQHYSI
PLTFGQGTKLEIK.

In particular embodiments, the anti-DLK1 antibody includes a variable heavy chain including a complementarity determining region (CDR) heavy 1 (CDRH1) including the sequence GFSLSIYS (SEQ ID NO: 47), a CDRH2 including the sequence IWGGGST (SEQ ID NO: 48), and a CDRH3 including the sequence ARKEGNYLWFAY (SEQ ID NO: 49), and a variable light chain including a CDR light 1 (CDRL1) including the sequence QSLLQSSNQKNY (SEQ ID NO: 50), a CDRL2 including the sequence FAS, and a CDRL3 including the sequence QQHYSIPLT (SEQ ID NO: 51).

In particular embodiments, the anti-DLK1 antibody includes a variable heavy chain including the sequence: QVQLQESGPGLVKPSETLSLTCTVSGFSLTSYGVSWVRQPPGKGLEWIGVIWGDGSTSYNPS LKSRVTISKDTSKNQVSLKLSSVTAADTAVYYC AKPDGPLGQGTLVTVSS (SEQ ID NO: 52) and a variable light chain variable including the sequence:

(SEQ ID NO: 53)
DIVMTQSPLSLPVTPGEPASISCRSSQSLVHINGNTYLHWYLQKPGQSPQ
LLIYKVSNRFSGVPDRFSGSGSGTDFTLKISRVEAEDVGVYYCSQTTHVP
WTFGQGTKLEIK.

In particular embodiments, the anti-DLK1 antibody includes a variable heavy chain including a CDRH1 including the sequence GFSISDYY (SEQ ID NO: 54), a CDRH2 including the sequence INYDGTNT (SEQ ID NO: 55), and a CDRH3 including the sequence VRSYYYYGMEY (SEQ ID NO: 56), and a variable light chain including a CDRL1 including the sequence HDVSTA (SEQ ID NO: 57), a CDRL2 including the sequence SAS, and a CDRL3 including the sequence QQHYRIPLT (SEQ ID NO: 58).

In particular embodiments, the DLK1 antagonist includes an oligonucleotide listed in Table 1. In particular embodiments, the DLK1 antagonist includes an oligonucleotide including the sequence of SEQ ID NO: 11 or SEQ ID NO: 12, that relate to a position in Dlk1 (sense) at bp 263-287 (in CDS of NM_003836.7). In particular embodiments, the DLK1 antagonist includes an oligonucleotide including the sequence selected from the group including SEQ ID NOs: 13, 14, 15, 16, 17, 18, 19, and 20, that are related to a position in Dlk1 at bp 1270-1610 (i.e. in 3′+3′UTR-exon 6 of NM_003836.7). In other embodiments, the DLK1 antagonist includes an oligonucleotide including the sequence of SEQ ID NO: 21 or SEQ ID NO: 22, that are related to a position of exon 5 in Dlk1. In other embodiments, the DLK1 antagonist includes an oligonucleotide including the sequence of SEQ ID NO: 23 or SEQ ID NO: 24, that are related to a position of exon 2 in Dlk1. In other embodiments, the DLK1 antagonist includes an oligonucleotide including the sequence of SEQ ID NO: 25 or SEQ ID NO: 26, that are related to a position of exon 5 in Dlk1. In other embodiments, the DLK1 antagonist includes an oligonucleotide including the sequence selected from the group including SEQ ID NOs: 27, 28, 29, 30, 31, and 32, that are related to a position of exon 6 in Dlk1.

TABLE 1
RNA/DNA for inhibition of human Dlk1. Target
sequence: Homo sapiens delta like non-canonical
Notch ligand 1 (Dlk1) of NCBI Reference Sequence:
NM_003836.7. Bases with underline refers to RNA
whereas bases in bold refers to DNA.

| | SEQ ID NO: for |
|---|---|
| | underlined portion |
| Sequence | of sequence |
| CCUGCAACCCCCAAAAUGGAUUCTG | 11 |
| CAGAAUCCAUUUUGGGGGUUGCAGGCC | 12 |
| CUCUCUUAAUGCAUGAUACAGAATA | 13 |
| UAUUCUGUAUCAUGCAUUAAGAGAGAG | 14 |
| CUUGUGUCAAAUCUGGUGAACGCTA | 15 |
| UAGCGUUCACCAGAUUUGACACAAGAG | 16 |
| CUCUCUCUUAAUGCAUGAUACAGAA | 17 |
| UUCUGUAUCAUGCAUUAAGAGAGAGAA | 18 |
| AAAUAAGUAUGUUAUUCUAAAAUCT | 19 |
| AGAUUUUAGAAUAACAUACUUAUUUCU | 20 |
| CUCUUAAUGCAUGAUACAGAAUAAT | 21 |
| AUUAUUCUGUAUCAUGCAUUAAGAGAG | 22 |
| AAAAUGGAUUCUGCGAGGAUGACAA | 23 |
| UUGUCAUCCUCGCAGAAUCCAUUUUGG | 24 |
| GGCUUCUCAGGCAAUUUCUGCGAGA | 25 |
| UCUCGCAGAAAUUGCCUGAGAAGCCAG | 26 |
| CUUAAUGCAUGAUACAGAATT | 27 |
| UUCUGUAUCAUGCAUUAAGAG | 28 |
| CAAACGCAAUGCAAAAACATT | 29 |
| UGUUUUUGCAUUGCGUUUGTC | 30 |
| GCUUACAUAUAUUGUCUUUTT | 31 |
| AAAGACAAUAUAUGUAAGCGT | 32 |

In particular embodiments, the DLK1 antagonist includes an oligonucleotide, wherein the oligonucleotide is a double stranded RNA including SEQ ID NO: 11 and SEQ ID NO: 12. In particular embodiments, the DLK1 antagonist includes an oligonucleotide, wherein the oligonucleotide is a double stranded RNA including SEQ ID NO: 13 and SEQ ID NO: 14. In particular embodiments, the DLK1 antagonist includes an oligonucleotide, wherein the oligonucleotide is a double stranded RNA including SEQ ID NO: 15 and SEQ ID NO: 16. In particular embodiments, the DLK1 antagonist includes an oligonucleotide, wherein the oligonucleotide is a double stranded RNA including SEQ ID NO: 17 and SEQ ID NO: 18. In particular embodiments, the DLK1 antagonist includes an oligonucleotide, wherein the oligonucleotide is a double stranded RNA including SEQ ID NO: 19 and SEQ ID NO: 10. In particular embodiments, the DLK1 antagonist includes an oligonucleotide, wherein the oligonucleotide is a double stranded RNA including SEQ ID NO: 21 and SEQ ID NO: 22. In particular embodiments, the DLK1 antagonist includes an oligonucleotide, wherein the oligonucleotide is a double stranded RNA including SEQ ID NO: 23 and SEQ ID NO: 24. In particular embodiments, the DLK1 antagonist includes an oligonucleotide, wherein the oligonucleotide is a double stranded RNA including SEQ ID NO: 25 and SEQ ID NO: 26. In particular embodiments, the DLK1 antagonist includes an oligonucleotide, wherein the oligonucleotide is a double stranded RNA including SEQ ID NO: 27 and SEQ ID NO: 28. In particular embodiments, the DLK1 antagonist includes an oligonucleotide, wherein the oligonucleotide is a double stranded RNA including SEQ ID NO: 29 and SEQ ID NO: 30. In particular embodiments, the DLK1 antagonist includes an oligonucleotide, wherein the oligonucleotide is a double stranded RNA including SEQ ID NO: 31 and SEQ ID NO: 32.

In particular embodiments, the DLK1 antagonist includes an siRNA. In particular embodiments, the siRNA targets Dlk1 mRNA splice variants. In particular embodiments, these siRNA include the sequence: AGAUCGUAGCCGCAACCAATT (SEQ ID NO: 59; for underlined portion of the sequence) or UUGGUUGCGGCUACGAUCUCA (SEQ ID NO: 60). In particular embodiments, the siRNA targets Dlk1 mRNA splice variants containing coding sequences for the protease site for extracellular cleavage. In particular embodiments, these siRNA include the sequence: UCCUGAAGGUGUCCAUGAATT (SEQ ID NO: 61; for underlined portion of the sequence) or UUCAUGGACACCUUCAGGATG (SEQ ID NO: 62; for underlined portion of the sequence).

In particular embodiments, a DLK1 antagonist includes periostin.

In certain examples, the activity modulators are inhibitors that provide DLK1 modifications that reduce the natural function of DLK1. Particular embodiments modify one or more of Arg160, Gly199, Arg267, and/or Val272 in the ECD; pi-pi interacting residues 171, 173, 174, 186, and/or 187; one or more signal peptide residues within residues 1-23; and/or one or more of the 36 cysteine residues within the ECD. Cysteine residues outside of the ECD may also be modified.

Particular embodiments mutate (e.g., substitute or delete) Arg160. Particular embodiments mutate (e.g., substitute or delete) Gly199. Particular embodiments mutate (e.g., substitute or delete) Arg267. Particular embodiments mutate (e.g., substitute or delete) Val272. Particular embodiments mutate (e.g., substitute or delete) Arg160 and Gly199. Particular embodiments mutate (e.g., substitute or delete) Arg160 and Arg267. Particular embodiments mutate (e.g., substitute or delete) Arg160 and Val272. Particular embodiments mutate (e.g., substitute or delete) Gly199 and Arg267. Particular embodiments mutate (e.g., substitute or delete) Gly199 and Val272. Particular embodiments mutate (e.g., substitute or delete) Arg267 and Val272. Antibodies against single or combination of disease linked mutations occurring in the ECD of DLK1 can also be used.

(ii) DLK1 Condensate-Modifying Therapeutics. In particular embodiments, DLK1 condensate-modifying therapeutics include any substance, molecule, or compound that increases or decreases signaling through DLK1 condensates.

In particular embodiments, DLK1 condensates include DLK1 or fragments thereof. In particular embodiments, DLK1 condensates include a TGFβ activity modulator, a gamma secretase complex modulator, and/or an integrin activity modulator. In particular embodiments, the TGFβ activity modulator includes TGFβ receptor 1 (TGFβR1), TGFβR2, endoglin (ENG), or activin receptor type IB (ACVR1B). In particular embodiments, the gamma secretase complex modulator cleaves Notch, tumor necrosis factor receptor superfamily member 1A (TNFR1), or CD44. In particular embodiments, the gamma secretase complex modulator cleaves Notch 1. In particular embodiments, the gamma secretase complex modulator includes PSEN1. In particular embodiments, the integrin activity modulator includes α2 subunit of integrin αIIbβ3 (ITGA2B), β2 subunit of integrin (ITGB2), or integrin subunit αL (ITGAL).

In particular embodiments, DLK1 activity inhibits or lowers TGFβ signaling and/or Notch cleavage or signaling. DLK1 interacts with receptor components (e.g., TGFβ signaling components, cleavers of gamma secretase complex, and/or integrins) to impact receptor dynamics (e.g., assembly, trafficking, and/or turnover). DLK1 inhibits receptor signaling pathways by impairing receptor dynamics during phase separation. As such, the effects of DLK1 can be enhanced or perturbed by modulating these signaling pathways. For example, in particular embodiments, the effects of DLK1 (e.g., stem cell quiescence and longer-term repopulation) can be increased by administering a TGFβ activity modulator, a gamma secretase complex modulator, and/or a integrin activity modulator that decreases TGFβ activity, gamma secretase complex cleavage or signaling, and/or integrin activity, respectively. In particular embodiments, the effects of DLK1 can be bypassed or minimized by increasing signaling through receptors in the DLK1 condensate by administering a TGFβ activity modulator, a gamma secretase complex modulator, and/or a integrin activity modulator that increases TGFβ activity, gamma secretase complex cleavage or signaling, and/or integrin activity, respectively.

In particular embodiments, the TGFβ activity modulator increases TGFβR1. In particular embodiments, the TGFβ activity modulator that increases TGFβR1 includes an TGFβR1 protein (e.g., PDB: 3KCF_A, PDB: 3KCF_B, PDB: 3KCF_C, PDB: 3KCF_D, PDB: 3KCF_E) or variant, fragment, or encoding sequence thereof. In particular embodiments, the TGFβ activity modulator that increases TGFβR1 includes any substance, molecule, or compound that increases TGFβR1 activity. In particular embodiments, the TGFβ activity modulator that decreases TGFβR1 includes galunisertib (LY2157299), AZ12601011, AZ12799734, or SB-525334. In particular embodiments, the TGFβ activity modulator that decreases TGFβR1 includes any substance, molecule, or compound that decreases TGFβR1 activity, such as a protein inhibitor (e.g., antibody), a small molecule inhibitor, an interfering RNA (e.g., siRNA, shRNA), or a decoy.

In particular embodiments, the TGFβ activity modulator increases TGFβR2. In particular embodiments, the TGFβ activity modulator that increases TGFβR2 includes an TGFβR2 protein (e.g., UniProtKB/Swiss-Prot: P37173.2) or variant, fragment, or encoding sequence thereof. In particular embodiments, the TGFβ activity modulator that increases TGFβR2 includes any substance, molecule, or compound that increases TGFβR2 activity. In particular embodiments, the TGFβ activity modulator that decreases TGFβR2 includes TA-02, LY2109761, or LY364947. In particular embodiments, the TGFβ activity modulator that decreases TGFβR2 includes any substance, molecule, or compound that decreases TGFβR2 activity, such as a protein inhibitor (e.g., antibody), a small molecule inhibitor, an interfering RNA (e.g., siRNA, shRNA), or a decoy.

In particular embodiments, the TGFβ activity modulator increases endoglin. In particular embodiments, the TGFβ activity modulator that increases endoglin includes an endoglin protein (e.g., GenBank: AAC63386.1) or variant, fragment, or encoding sequence thereof. In particular embodiments, the TGFβ activity modulator that increases endoglin includes any substance, molecule, or compound that increases endoglin activity. In particular embodiments, the TGFβ activity modulator that decreases endoglin includes carotuximab (TRC105). In particular embodiments, the TGFβ activity modulator that decreases endoglin includes any substance, molecule, or compound that decreases endoglin activity, such as a protein inhibitor (e.g., antibody), a small molecule inhibitor, an interfering RNA (e.g., siRNA, shRNA), or a decoy.

In particular embodiments, the TGFβ activity modulator increases ACVR1B. In particular embodiments, the TGFβ activity modulator that increases ACVR1B includes an ACVR1B protein (e.g., NCBI Reference Sequence: NP_004293.1, NP_001399708.1, NP_001399703.1, NP_001399704.1, NP_001399705.1, NP_001399706.1, NP_001399711.1, NP_001399714.1, or NP_001399715.1) or variant, fragment, or encoding sequence thereof. In particular embodiments, the TGFβ activity modulator that increases ACVR1B includes any substance, molecule, or compound that increases ACVR1B activity. In particular embodiments, the TGFβ activity modulator that decreases ACVR1B includes any substance, molecule, or compound that decreases ACVR1B activity, such as a protein inhibitor (e.g., antibody), a small molecule inhibitor, an interfering RNA (e.g., siRNA, shRNA), or a decoy.

In particular embodiments, the gamma secretase complex modulator increases Notch cleavage. In particular embodiments, the gamma secretase complex modulator that increases Notch cleavage includes a PSEN1 protein (e.g., GenBank: AAH11729.1) or variant, fragment, or encoding sequence thereof. In particular embodiments, the gamma secretase complex modulator that increases Notch cleavage includes a PSEN2 protein (e.g., GenBank: AAH06365.1) or variant, fragment, or encoding sequence thereof. In particular embodiments, the gamma secretase complex modulator that increases Notch cleavage includes any substance, molecule, or compound that increases Notch cleavage. In particular embodiments, the gamma secretase complex modulator that decreases Notch cleavage includes the PSEN1 inhibitor MRK-560 or compound 13K. In particular embodiments, the gamma secretase complex modulator that decreases Notch cleavage includes the PSEN2 inhibitor 4-aminoquinolines or imidazole. In particular embodiments, the gamma secretase complex modulator that decreases Notch cleavage includes any substance, molecule, or compound that decreases Notch cleavage, such as a protein inhibitor (e.g., antibody), a small molecule inhibitor, an interfering RNA (e.g., siRNA, shRNA), or a decoy. In particular embodiments, the gamma secretase complex modulator includes the Notch inhibitor DAPT.

In particular embodiments, the gamma secretase complex modulator increases TNFR1 cleavage. In particular embodiments, the gamma secretase complex modulator that increases TNFR1 cleavage includes a zinc metalloprotease protein (e.g., TNFα-converting enzyme (TACE) (UniProtKB/Swiss-Prot: P78536.1)) or variant, fragment, or encoding sequence thereof. In particular embodiments, the gamma secretase complex modulator that increases TNFR1 cleavage includes any substance, molecule, or compound that increases TNFR1 cleavage. In particular embodiments, the gamma secretase complex modulator that decreases TNFR1 cleavage includes a TACE inhibitor (e.g., TAPI-2, TAPI-O, TAPI-1). In particular embodiments, the gamma secretase complex modulator that decreases TNFR1 cleavage includes any substance, molecule, or compound that decreases TNFR1 cleavage, such as a protein inhibitor (e.g., antibody), a small molecule inhibitor, an interfering RNA (e.g., siRNA, shRNA), or a decoy.

In particular embodiments, the gamma secretase complex modulator increases CD44 cleavage. In particular embodiments, the gamma secretase complex modulator that increases CD44 cleavage includes a membrane-associated metalloproteases like membrane-type 1 matrix metalloproteinase (MT-1-MMP (e.g., UniProtKB/Swiss-Prot: P50281.3), a disintegrin and metalloproteinase domain (ADAM) 10 (e.g., GenBank: AAC51766.1), ADAM17 (also referred to as TACE; e.g., GenBank: AAI46659.1 or GenBank: AAH62687.1) or variants, fragments, or encoding sequences thereof. In particular embodiments, the gamma secretase complex modulator that increases CD44 cleavage includes any substance, molecule, or compound that increases CD44 cleavage. In particular embodiments, the gamma secretase complex modulator that decreases CD44 cleavage includes an MMP14 inhibitor (e.g., clioquinol, DX-2400, hemopexin domain mimetics of MMP14), an ADAM10 inhibitor (e.g., GI 254023X), or an ADAM17 inhibitor (e.g., TAPI-1, TAPI-0, TAPI-2). In particular embodiments, the gamma secretase complex modulator that decreases CD44 cleavage includes any substance, molecule, or compound that decreases CD44 cleavage, such as a protein inhibitor (e.g., antibody), a small molecule inhibitor, an interfering RNA (e.g., siRNA, shRNA), or a decoy.

In particular embodiments, the integrin activity modulator increases ITGA2B. In particular embodiments, the integrin activity modulator that increases ITGA2B includes an IGA2B protein (e.g., UniProtKB/Swiss-Prot: P08514.3) or variant, fragment, or encoding sequence thereof. In particular embodiments, the integrin activity modulator that increases ITGA2B includes any substance, molecule, or compound that increases ITGA2B activity. In particular embodiments, the integrin activity modulator that decreases ITGA2B includes abciximab, eptifibatide, and tirofiban. In particular embodiments, the integrin activity modulator that decreases ITGA2B includes any substance, molecule, or compound that decreases ITGA2B activity, such as a protein inhibitor (e.g., antibody), a small molecule inhibitor, an interfering RNA (e.g., siRNA, shRNA), or a decoy.

In particular embodiments, the integrin activity modulator increases ITGB2. In particular embodiments, the integrin activity modulator that increases ITGB2 includes an ITGB2 protein (e.g., GenBank: AAH05861.1) or variant, fragment, or encoding sequence thereof. In particular embodiments, the integrin activity modulator that increases ITGB2 includes any substance, molecule, or compound that increases ITGB2 activity. In particular embodiments, the integrin activity modulator that decreases ITGB2 includes erlizumab. In particular embodiments, the integrin activity modulator that decreases ITGB2 includes any substance, molecule, or compound that decreases ITGB2 activity, such as a protein inhibitor (e.g., antibody), a small molecule inhibitor, an interfering RNA (e.g., siRNA, shRNA), or a decoy.

In particular embodiments, the integrin activity modulator increases ITGAL. In particular embodiments, the integrin activity modulator that increases ITGAL includes an ITGAL protein (e.g., GenBank: AAH08777.1) or variant, fragment, or encoding sequence thereof. In particular embodiments, the integrin activity modulator that increases ITGAL includes any substance, molecule, or compound that increases ITGAL activity. In particular embodiments, the integrin activity modulator that decreases ITGAL includes efalizumab. In particular embodiments, the integrin activity modulator that decreases ITGAL includes any substance, molecule, or compound that decreases ITGAL activity, such as a protein inhibitor (e.g., antibody), a small molecule inhibitor, an interfering RNA (e.g., siRNA, shRNA), or a decoy.

(iii) Genetic Engineering Techniques. In particular embodiments, cells are genetically modified to overexpress or downregulate DLK1. In particular embodiments, cells are genetically modified to overexpress or downregulate DLK1-related pathways (e.g., TGFβ or Notch signaling pathways).

Desired genes (e.g., genes encoding DLK1 modulators) or artificial expression constructs disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

The term “gene” refers to a nucleic acid sequence (used interchangeably with polynucleotide or nucleotide sequence) that encodes a DLK1 modulator as described herein. This definition includes various sequence polymorphisms, mutations, and/or sequence variants wherein such alterations do not substantially affect the function of the encoded DLK1 modulator. The term “gene” may include not only coding sequences but also regulatory regions such as promoters, enhancers, and termination regions. The term further can include all introns and other DNA sequences spliced from an mRNA transcript, along with variants resulting from alternative splice sites. Gene sequences encoding the molecule can be DNA or RNA that directs the expression of the open reading frame(s) within the artificial expression construct. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. Portions of complete gene sequences are referenced throughout the disclosure as is understood by one of ordinary skill in the art. Herein, the term “gene” and “artificial expression construct” are used interchangeably.

Gene sequences encoding a DLK1 modulator are provided herein and can also be readily prepared by synthetic or recombinant methods from the relevant amino acid sequences and other description provided herein. In embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In embodiments, the gene sequence encoding the sequences can be codon optimized for expression in mammalian cells.

“Encoding” refers to the property of specific sequences of nucleotides in a gene, such as a cDNA, or an mRNA, to serve as templates for the synthesis of other macromolecules such as a defined sequence of amino acids. Thus, a gene codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function.

Polynucleotide gene sequences encoding more than one portion of an expressed DLK1 modulator can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

Promoters can include general promoters, tissue-specific promoters, cell-specific promoters, and/or promoters specific for the cytoplasm. Promoters may include strong promoters, weak promoters, constitutive expression promoters (i.e., constitutive promoters), and/or inducible promoters. Constitutive promoters are promoters that allow for the continual transcription of its associated gene or genes.

Particular examples of constitutive promoters include human elongation factor Iα (EF1α) promoter, myeloproliferative sarcoma virus (MND), cytomegalovirus (CMV), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as the actin promoter, the myosin promoter, the hemoglobin promoter, and the creatine kinase promoter. In particular embodiments, the artificial expression construct is under the control of an EF1 promoter.

A “vector” is a nucleic acid molecule that is capable of transporting another nucleic acid. Vectors may be, e.g., plasmids (DNA plasmids or RNA plasmids), transposon-based systems, cosmids, bacterial artificial chromosomes, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

A lentiviral vector is a vector derived from at least a portion of a lentivirus genome, including especially a self-inactivating lentiviral vector as provided in Milone et ah, Mol. Ther. 17(8): 1453-1464 (2009). Other examples of lentivirus vectors that may be used in the clinic, include: the LENTIVECTOR® gene delivery technology from Oxford BioMedica, the LENTIMAX™ vector system from Lentigen and the like. Nonclinical types of lentiviral vectors are also available and would be known to one skilled in the art. In particular embodiments, cells are genetically engineered to express a DLK1 modulator using a lentivirus or lentiviral vector.

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

Retroviral vectors (see Miller, et al., 1993, Meth. Enzymol. 217:581-599) can be used. In such embodiments, the gene to be expressed is cloned into the retroviral vector for its delivery into cells. In particular embodiments, a retroviral vector includes all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Adenoviruses, adeno-associated viruses (AAV) and alphaviruses can also be used. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19:673-686. Other methods of gene delivery include use of mammalian artificial chromosomes (Vos, 1998, Curr. Op. Genet. Dev. 8:351-359); liposomes (Tarahovsky and Ivanitsky, 1998, Biochemistry (Mosc) 63:607-618); ribozymes (Branch and Klotman, 1998, Exp. Nephrol. 6:78-83); and triplex DNA (Chan and Glazer, 1997, J. Mol. Med. 75:267-282).

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including transgenes are described in, e.g., U.S. Pat. No. 8,119,772; Walchli, et al., 2011, PLoS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. ZFNs are used to introduce double stranded breaks (DSBs) at a specific site in a DNA sequence which enables the ZFNs to target unique sequences within a genome in a variety of different cells. A zinc finger is a domain of 30 amino acids within the zinc finger binding domain whose structure is stabilized through coordination of a zinc ion. Examples of zinc fingers include C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. A well-known example of a ZFN is a fusion of the FokI nuclease with a zinc finger DNA binding domain. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing double DSBs in the DNA, which induce repair mechanisms in cells. Generally, two TALENs must bind and flank each side of the target DNA site for the DNA cleavage domain to dimerize and induce a DSB. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(I):49-55; Beurdeley et al., Nat Commun, 2013, 4:1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments can use transposon-based systems as gene editing agents to mediate the integration of an artificial expression construct into cells. Generally, such methods will involve introducing into cells (i) a first vector encoding a transposase (or a transposase polypeptide) and (ii) a second vector encoding a desired genetic element that is flanked by transposon repeats. Transposons or transposable elements include a (short) nucleic acid sequence with terminal repeat sequences upstream and downstream thereof and encode enzymes that facilitate the excision and insertion of the nucleic acid into target DNA sequences.

Several transposon/transposase systems have been adapted for genetic insertions of heterologous DNA sequences. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON. Transposases and transposon systems are further described in U.S. Pat. Nos. 6,489,458; 7,148,203; 8,227,432; and 9,228,180.

(iv) Compositions for Administration. DLK1 modulators (e.g., DLK1 inhibitors, DLK1 expression constructs, DLK1 ECD, DLK1 peptides, antibodies) and DLK1 condensate-modifying therapeutics can be formulated into compositions for administration to subjects or for administration to cells in vitro. Salts and/or pro-drugs of DLK1 modulators can also be used. A pharmaceutically acceptable salt includes any salt that retains the activity of a DLK1 modulator and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or hydrolysis of a biologically labile group.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes DLK1 modulators or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the composition can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot compositions can include a variety of bioerodible polymers including poly(lactide), poly(glycolide), poly(caprolactone) and poly(lactide)-co(glycolide) (PLG) of desirable lactide: glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl) methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of nanoparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

In particular embodiments, the compositions include DLK1 modulators making up at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, compositions and formulations can be prepared to meet sterility, pyrogenicity, general safety, and purity standards as required by U.S. FDA Office of Biological Standards and/or other relevant foreign regulatory agencies.

(v) Formulations. In particular embodiments, cells (e.g., genetically-modified cells) can be harvested from a culture medium and washed and concentrated into a carrier in a therapeutically-effective amount. Exemplary carriers include saline, buffered saline, physiological saline, water, Hanks' solution, Ringer's solution, Nonnosol-R (Abbott Labs), PLASMA-LYTE A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Where necessary or beneficial, compositions or formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In formulations disclosed herein, cells are generally in a volume of a liter or less, 500 mls or less, 250 mls or less or 100 mls or less. Hence the density of administered cells is typically greater than 10⁴ cells/ml, 10⁷ cells/ml or 10⁸ cells/ml.

(vi) Methods of Use. Particular methods disclosed herein include treating subjects. Subjects include, e.g., humans, veterinary animals (dogs, cats, reptiles, birds) livestock (e.g., horses, cattle, goats, pigs, chickens) and research animals (e.g., monkeys, rats, mice, fish). Treating subjects includes delivering therapeutically effective amounts. Therapeutically effective amounts include those that provide effective amounts, prophylactic treatments and/or therapeutic treatments.

An “effective amount” is the amount of a composition or formulation necessary to result in a desired physiological change in the subject. Effective amounts are often administered for research purposes. Effective amounts disclosed herein can cause a statistically-significant effect in an animal model or in vitro assay relevant to the assessment of a condition's development, progression, and/or resolution.

A “prophylactic treatment” includes a treatment administered to a subject who does not display signs or symptoms of a condition or displays only early signs or symptoms of a condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment, or therapeutic treatment are not mutually exclusive, and in particular embodiments, administered dosages may accomplish more than one treatment type.

In particular embodiments, therapeutically effective amounts of DLK1 modulators that decrease DLK1 activity shifts stem cells (e.g., HSC) from a quiescent to an activated state. In particular embodiments, DLK1 knockdown leads to loss of HSPC repopulating activity. In particular embodiments, DLK1 knockdown leads to enhanced myeloid differentiation of HSPC.

In particular embodiments, therapeutically effective amounts of DLK1 modulators that increase DLK1 activity maintains stem cell quiescence. In particular embodiments, therapeutically effective amounts of DLK1 modulators that increase DLK1 activity delays stem cell differentiation. In particular embodiments, therapeutically effective amounts of DLK1 modulators that increase DLK1 activity enhances longer-term repopulation of stem cells.

In particular embodiments, DLK1 overexpression leads to a delay in CD34+ cell differentiation. In particular embodiments, DLK1 overexpression leads to an increased number of CD34+CD90^{lo} HSPC and enhanced longer-term repopulation. In particular embodiments, exposure to DLK1 ECD leads to an increased number of CD34+CD90^{lo} HSPC and enhanced longer-term repopulation.

In particular embodiments, therapeutically effective amounts of DLK1 condensate-modifying therapeutics can enhance or perturb the effects of DLK1 as described elsewhere herein.

In certain examples, formulations including cells disclosed herein with modulated DLK1 activity can be administered to a subject to reconstitute the subject's immune system after, for example, a conditioning therapy, as part of a stem cell transplant, or after exposure to radiation.

In particular embodiments of methods, the modulated DLK1 activity is upregulated DLK1 activity and the subject has received or is receiving chemotherapy or another cytotoxic agent.

In particular embodiments of methods, the modulated DLK1 activity is downregulated DLK1 activity and the subject has clonal hematopoiesis.

In particular embodiments of methods, the DLK1 modulator upregulates DLK1 activity and the subject has received or is receiving chemotherapy or another cytotoxic agent.

In particular embodiments of methods, the DLK1 modulator downregulates DLK1 activity and the subject has clonal hematopoiesis.

Exemplary methods to determine effects described herein are provided in the Experimental Examples section or are known to those of ordinary skill in the art.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (VII) EXEMPLARY EMBODIMENTS

1. A method of delaying stem cell differentiation within a population of stem cells including administering a delta-like non-canonical Notch ligand 1 (DLK1) agonist to the population of stem cells, thereby delaying stem cell differentiation.
2. The method of embodiment 1, wherein the population of stem cells include hematopoietic stem/progenitor cells (HSPC).
3. The method of embodiment 1 or 2, wherein the population of stem cells include hematopoietic stem cells (HSC).
4. The method of any of embodiments 1-3, wherein the DLK1 agonist increases DLK1 expression and/or DLK1 activity.
5. The method of any of embodiments 1-4, wherein the DLK1 agonist includes a DLK1 protein, a fragment of a DLK1 protein, an anti-DLK1 antibody, a DLK1 mimetic, a nucleic acid sequence encoding DLK1, a nucleic acid sequence encoding a fragment of DLK1, a nucleic acid sequence encoding an anti-DLK1 antibody, or a nucleic acid sequence that binds SEQ ID NO: 40.
6. The method of embodiment 5, wherein the DLK1 protein includes the sequence of SEQ ID NO: 1 or a sequence having at least 90% sequence identity to SEQ ID NO: 1.
7. The method of embodiment 5 Or 6, wherein the fragment of the DLK1 protein includes an extracellular domain of the DLK1 protein.
8. The method of any of embodiments 5-7, wherein the fragment of the DLK1 protein includes the sequence of SEQ ID NO: 2 or any of SEQ ID NOs 36-39 or a sequence having at least 90% sequence identity to the sequence of SEQ ID NO: 2 or any of SEQ ID NOs 36-39.
9. The method of any of embodiments 5-8, wherein the nucleic acid sequence encoding DLK1 includes the sequence of SEQ ID NO: 35 or a sequence having at least 90% sequence identity to the sequence of SEQ ID NO: 35.
10. The method of any of embodiments 5-9, wherein the nucleic acid sequence encoding the fragment of DLK1 includes a fragment of the sequence set forth in SEQ ID NO: 35 or a sequence having at least 90% sequence identity to the sequence of SEQ ID NO: 35.
11. The method of any of embodiments 5-10, wherein the nucleic acid sequence that binds SEQ ID NO: 40 has the sequence of SEQ ID NO: 41 or SEQ ID NO: 42.
12. The method of any of embodiments 5-11, wherein the nucleic acid sequence is within a vector.
13. The method of embodiment 12, wherein the vector includes a lentivirus vector.
14. The method of any of embodiments 1-13, wherein the DLK1 agonist decreases TGFβ signaling and/or Notch cleavage.
15. The method of any of embodiments 1-14, wherein the delaying stem cell differentiation includes maintaining stem cell quiescence.
16. The method of any of embodiments 1-15, wherein the delaying stem cell differentiation includes longer-term repopulation of stem cells.
17. The method of any of embodiments 1-16, wherein the delaying stem cell differentiation includes a delay in CD34+ cell differentiation.
18. The method of any of embodiments 1-17, wherein the delaying stem cell differentiation results in an increased number of CD34+CD90lo HSPC.
19. The method of any of embodiments 1-18, wherein the population of stem cells are ex vivo stem cells.
20. The method of embodiment 19, wherein the ex vivo stem cells are for adoptive cell transfer.
21. The method of any of embodiments 1-20, further including genetically modifying the population of stem cells, expanding the genetically modified stem cells, and shifting the stem cells to a quiescent state for transplantation.
22. The method of any of embodiments 1-21, wherein the population of stem cells are used in a gene therapy.
23. The method of any of embodiments 1-22, further including culturing the population of stem cells on endothelial cells.
24. The method of embodiment 23, wherein the culturing further includes incubation in media containing growth factors selected from stem cell factor (SCF), FMS-like tyrosine kinase 3 ligand (FLT3L), thrombopoietin (TPO), interleukin (IL)-6, and/or IL-3.
25. The method of any of embodiments 1-24, wherein the population of stem cells are within a subject.
26. A method of shifting a population of stem cells from an activated state to a quiescent state including administering a delta-like non-canonical Notch ligand 1 (DLK1) agonist to the population of stem cells, thereby shifting the population of stem cells to the quiescent state.
27. The method of embodiment 26, wherein the population of stem cells include hematopoietic stem/progenitor cells (HSPC).
28. The method of embodiment 26 or 27, wherein the population of stem cells include hematopoietic stem cells (HSC).
29. The method of any of embodiments 26-28, wherein the DLK1 agonist increases DLK1 expression and/or DLK1 activity.
30. The method of any of embodiments 26-29, wherein the DLK1 agonist includes a DLK1 protein, a fragment of a DLK1 protein, an anti-DLK1 antibody, a DLK1 mimetic, a nucleic acid sequence encoding DLK1 a nucleic acid sequence encoding a fragment of DLK1, a nucleic acid sequence encoding an anti-DLK1 antibody, or a nucleic acid sequence that binds SEQ ID NO: 40.
31. The method of embodiment 30, wherein the DLK1 protein includes the sequence of SEQ ID NO: 1 or a sequence having at least 90% sequence identity to SEQ ID NO: 1.
32. The method of embodiment 30, wherein the fragment of the DLK1 protein includes an extracellular domain of the DLK1 protein.
33. The method of any of embodiments 30-32, wherein the fragment of the DLK1 protein includes the sequence of SEQ ID NO: 2 or any of SEQ ID NOs 36-39.
34. The method of any of embodiments 30-33, wherein the nucleic acid sequence encoding DLK1 includes the sequence of SEQ ID NO: 35 or a sequence having at least 90% sequence identity to the sequence of SEQ ID NO: 35.
35. The method of any of embodiments 30-34, wherein the nucleic acid sequence encoding the fragment of DLK1 includes a fragment of the sequence set forth in SEQ ID NO: 35 or a sequence having at least 90% sequence identity to the sequence of SEQ ID NO: 35.
36. The method of any of embodiments 30-35, wherein the nucleic acid sequence that binds SEQ ID NO: 40 has the sequence of SEQ ID NO: 41 or SEQ ID NO: 42.
37. The method of any of embodiments 30-36, wherein the nucleic acid sequence is within a vector.
38. The method of embodiment 37, wherein the vector includes a lentivirus vector.
39. The method of any of embodiments 26-38, wherein the DLK1 agonist decreases TGFβ signaling and/or Notch cleavage.
40. The method of any of embodiments 26-39, wherein the shifting the population of stem cells to the quiescent state includes delaying stem cell differentiation.
41. The method of any of embodiments 26-40, wherein the shifting the population of stem cells to the quiescent state includes longer-term repopulation of stem cells.
42. The method of any of embodiments 26-41, wherein the population of stem cells are ex vivo stem cells.
43. The method of embodiment 42, wherein the ex vivo stem cells are for adoptive cell transfer.
44. The method of any of embodiments 26-43, further including genetically modifying the population of stem cells, and expanding the genetically modified stem cells, before the shifting the stem cells to the quiescent state.
45. The method of embodiment 44, wherein the population of stem cells are used in a gene therapy.
46. The method of any of embodiments 26-45, further including culturing the population of stem cells on endothelial cells.
47. The method of embodiment 46, wherein the culturing further includes incubation in media containing growth factors selected from SCF, FLT3L, TPO, IL-6, and/or IL-3.
48. The method of any of embodiments 26-47, wherein the population of stem cells are within a subject.
49. A method of shifting stem cells from a quiescent to activated state including contacting stem cells with a delta-like non-canonical Notch ligand 1 (DLK1) antagonist, thereby shifting stem cells to the activated state.
50. The method of embodiment 49, wherein the stem cells include hematopoietic stem/progenitor cells (HSPC).
51. The method of embodiment 49 or 50, wherein the stem cells include hematopoietic stem cells (HSC).
52. The method of any of embodiments 49-51, wherein the DLK1 antagonist decreases DLK1 expression and/or DLK1 activity.
53. The method of any of embodiments 49-52, wherein the DLK1 antagonist includes a DLK1 inhibitor.
54. The method of any of embodiments 49-53, wherein the DLK1 antagonist includes an anti-DLK1 antibody.
55. The method of embodiment 54, wherein the anti-DLK1 antibody includes a variable heavy chain including the sequence of SEQ ID NO: 43 and a variable light chain including the sequence of SEQ ID NO: 44.
56. The method of embodiment 54 or 55, wherein the anti-DLK1 antibody includes a variable heavy chain including the sequence of SEQ ID NO: 45 and a variable light chain including the sequence of SEQ ID NO: 46.
57. The method of any of embodiments 54-56, wherein the anti-DLK1 antibody includes a variable heavy chain including the sequence of SEQ ID NO: 53 and a variable light chain including the sequence of SEQ ID NO: 54.
58. The method of any of embodiments 49-57, wherein the DLK1 antagonist includes an oligonucleotide.
59. The method of embodiment 58, wherein the oligonucleotide includes any of SEQ ID NOs: 11-32, or 40.
60. The method of any of embodiments 49-59, wherein the DLK1 antagonist includes siRNA.
61. The method of embodiment 60, wherein the siRNA includes SEQ ID NO: 33, SEQ ID NO: 59, or any one of SEQ ID NOs: 60-62.
62. The method of any of embodiments 49-61, wherein the DLK1 antagonist includes a DLK1 decoy.
63. The method of any of embodiments 49-62, wherein the DKL1 antagonist biases the stem cells towards a myeloid lineage.
64. The method of any of embodiments 49-63, wherein the DLK1 antagonist increases TGFβ signaling and/or Notch cleavage.
65. The method of any of embodiments 49-64, wherein the stem cells are ex vivo stem cells.
66. The method of embodiment 65, wherein the ex vivo stem cells are for adoptive cell transfer.
67. The method of any of embodiments 49-64, wherein the stem cells are within a subject.
68. A method of enhancing DLK1 activity in stem cells including administering a DLK1 modulator or a DLK1 condensate modifying therapeutic, thereby enhancing DLK1 activity.
69. The method of embodiment 68, wherein the DLK1 modulator includes a DLK1 agonist.
70. The method of embodiment 69, wherein the DLK1 agonist includes a DLK1 protein, a fragment of a DLK1 protein, an anti-DLK1 antibody, a DLK1 mimetic, a nucleic acid sequence encoding DLK1, a nucleic acid sequence encoding a fragment of DLK1, a nucleic acid sequence encoding an anti-DLK1 antibody, or a nucleic acid sequence that binds SEQ ID NO: 40.
71. The method of embodiment 70, wherein the DLK1 protein includes the sequence of SEQ ID NO: 1 or a sequence having at least 90% sequence identity to SEQ ID NO: 1.
72. The method of embodiment 70 or 71, wherein the fragment of the DLK1 protein includes an extracellular domain of the DLK1 protein.
73. The method of any of embodiments 70-72, wherein the fragment of the DLK1 protein includes the sequence of SEQ ID NO: 2 or any of SEQ ID NOs 36-39.
74. The method of any of embodiments 70-73, wherein the nucleic acid sequence encoding DLK1 includes the sequence of SEQ ID NO: 35 or a sequence having at least 90% sequence identity to the sequence of SEQ ID NO: 35.
75. The method of any of embodiments 70-74, wherein the nucleic acid sequence encoding the fragment of DLK1 includes a fragment of the sequence set forth in SEQ ID NO: 35 or a sequence having at least 90% sequence identity to the sequence of SEQ ID NO: 35.
76. The method of any of embodiments 70-75, wherein the nucleic acid sequence that binds SEQ ID NO: 40 has the sequence of SEQ ID NO: 41 or SEQ ID NO: 42.
77. The method of any of embodiments 70-76, wherein the nucleic acid sequence is within a vector.
78. The method of embodiment 77, wherein the vector includes a lentivirus vector.
79. The method of any of embodiments 68-78, wherein the DLK1 condensate modifying therapeutic includes a TGFβ activity modulator, a gamma secretase complex modulator, or an integrin activity modulator.
80. The method of embodiment 79, wherein the TGFβ activity modulator includes a TGFβ signaling inhibitor.
81. The method of embodiment 79 or 80, wherein the gamma secretase complex modulator includes a Notch, a tumor necrosis factor receptor superfamily member 1A (TNFR1) cleavage inhibitor, or a CD44 cleavage inhibitor.
82. The method of any of embodiments 79-81, wherein the gamma secretase complex modulator includes a Notch1 cleavage inhibitor.
83. The method of any of embodiments 79-82, wherein the integrin activity modulator includes an ITGA2B inhibitor, ITGB2 inhibitor, or ITGAL inhibitor.
84. The method of any of embodiments 69-83, wherein the enhancing DLK1 activity occurs in vivo or ex vivo.
85. The method of any of embodiments 69-84, wherein the enhancing DLK1 activity shifts stem cells from an activated state to a quiescent state.
86. The method of any of embodiments 69-85, wherein the enhancing DLK1 activity allows longer-term repopulating of stem cells.
87. The method of any of embodiments 69-86, wherein the stem cells include hematopoietic stem/progenitor cells (HSPC).
88. The method of any of embodiments 69-87, wherein the stem cells include hematopoietic stem cells.
89. A method of ameliorating a hematopoietic stem cell (HSC) aging process including administering a DLK1 activity modulator or DLK1 condensate-modifying therapeutic, thereby ameliorating the aging process.
90. The method of embodiment 89, wherein the DLK1 activity modulator includes a DLK1 agonist.
91. The method of embodiment 89 or 90, wherein the DLK1 condensate-modifying therapeutic decreases TGFβ activity, gamma secretase complex cleavage, and/or integrin activity.
92. The method of any of embodiments 89-91, wherein the DLK1 activity modulators decrease differentiation of hematopoietic stem cells into mature myeloid precursors.
93. A method of decreasing DLK1 activity in stem cells including administering a DLK1 modulator or DLK1 condensate-modifying therapeutic, thereby decreasing DLK1 activity in stem cells.
94. The method of embodiment 93, wherein the DLK1 modulator includes a DLK1 antagonist.
95. The method of embodiment 94, wherein the DLK1 antagonist includes a DLK1 inhibitor.
96. The method of embodiment 94 or 95, wherein the DLK1 antagonist includes an anti-DLK1 antibody.
97. The method of embodiment 96, wherein the anti-DLK1 antibody includes a variable heavy chain including the sequence of SEQ ID NO: 43 and a variable light chain including the sequence of SEQ ID NO: 44.
98. The method of embodiment 96 or 97, wherein the anti-DLK1 antibody includes a variable heavy chain including the sequence of SEQ ID NO: 45 and a variable light chain including the sequence of SEQ ID NO: 46.
99. The method of any of embodiments 96-98, wherein the anti-DLK1 antibody includes a variable heavy chain including the sequence of SEQ ID NO: 53 and a variable light chain including the sequence of SEQ ID NO: 54.
100. The method of any of embodiments 94-99, wherein the DLK1 antagonist includes an oligonucleotide.
101. The method of embodiment 100, wherein the oligonucleotide includes any of SEQ ID NOs: 11-32 or 40.
102. The method of any of embodiments 94-101, wherein the DLK1 antagonist includes siRNA.
103. The method of embodiment 102, wherein the siRNA includes SEQ ID NO: 33, SEQ ID NO: 59, or any one of SEQ ID NOs: 60-62.
104. The method of any of embodiments 94-103, wherein the DLK1 antagonist includes a DLK1 decoy.
105. The method of any of embodiments 93-104, wherein the DLK1 condensate-modifying therapeutic includes a TGFβ activity modulator, a gamma secretase complex modulator, or an integrin activity modulator.
106. The method of embodiment 105, wherein the TGFβ activity modulator includes TGFβ receptor 1 (TGFβR1). TGFβR2, endoglin (ENG), or activin receptor type IB (ACVR1B).
107. The method of embodiment 105 or 106, wherein the gamma secretase complex modulator cleaves Notch, tumor necrosis factor receptor superfamily member 1A (TNFR1), or CD44.
108. The method of any of embodiments 105-107, wherein the gamma secretase complex modulator cleaves Notch1.
109. The method of any of embodiments 105-108, wherein the gamma secretase modulator includes PSEN1.
110. The method of any of embodiments 105-109, wherein the integrin activity modulator includes α2 subunit of integrin αIIbβ3 (ITGA2B), β2 subunit of integrin (ITGB2), or integrin subunit αL (ITGAL).
111. The method of any of embodiments 93-110, wherein the decreasing DLK1 activity occurs in vivo or ex vivo.
112. The method of any of embodiments 93-111, wherein the decreasing DLK1 activity shifts stem cells from a quiescent to activated state.
113. The method of any of embodiments 93-112, wherein the stem cells include hematopoietic stem/progenitor cells (HSPC).
114. The method of any of embodiments 93-113, wherein the stem cells include hematopoietic stem cells.
115. A formulation including stem cells with modulated DLK1 activity and a pharmaceutically acceptable carrier.
116. A method including administering the formulation of embodiment 115 to a subject.
117. The method of embodiment 116, wherein the modulated DLK1 activity is upregulated DLK1 activity and the subject has received or is receiving chemotherapy or another cytotoxic agent.
118. The method of embodiment 116, wherein the modulated DLK1 activity is downregulated DLK1 activity and the subject has clonal hematopoiesis.
119. A method of altering an activation state of stem cells within a subject, the method including administering a DLK1 modulator to the subject.
120. The method of embodiment 119, wherein the DLK1 modulator upregulates DLK1 activity and the subject has received or is receiving chemotherapy or another cytotoxic agent.
121. The method of embodiment 119, wherein the DLK1 modulator downregulates DLK1 activity and the subject has clonal hematopoiesis.

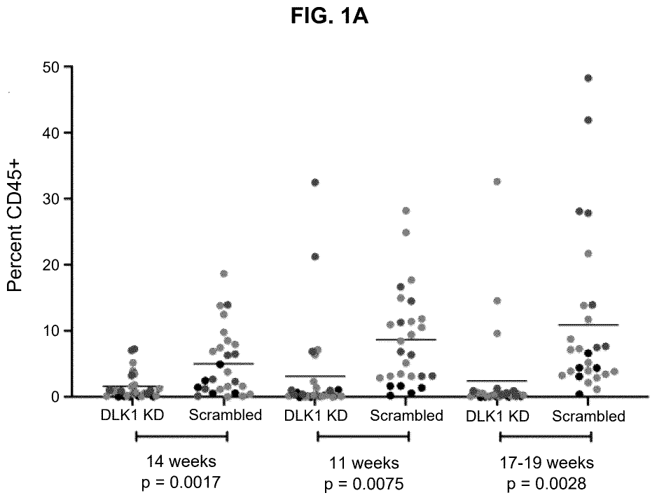

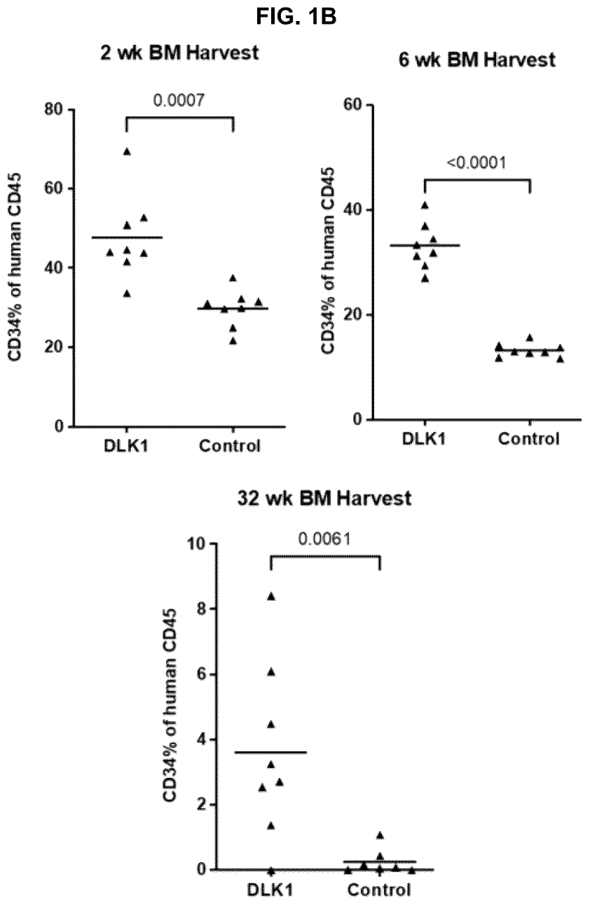

(viii) Experimental Examples. Experimental Example 1. Effect of DLK1 on hematopoietic stem cells (HSC). DLK1 knockdown reduces in vivo repopulation, whereas DLK1 overexpression delays in vivo CD34⁺ cell differentiation. To determine whether loss of DLK1 alters hematopoietic stem/progenitor cells (HSPC) function in vivo, the repopulating ability of cord blood (CB) CD34⁺ cells was assessed following DLK1 knockdown and DLK1 overexpression (FIGS. 1A, 1B). To do so, CB CD34⁺ cells were infected with lentiviruses encoding either DLK1 or scrambled control short hairpin RNAs (shRNAs) co-expressing GFP, a marker for viral integration (FIG. 1A) or DLK1-GFP or control GFP (FIG. 1B). 24 hours post-infection, cells from each group were transplanted into sub-lethally irradiated, immune deficient NOD-scid IL2Rgamma^{null} (NSG) mice. DLK1 knockdown significantly reduced both short (4 weeks) and longer-term (11 weeks; 17-19 weeks) repopulation of hematopoietic cells (FIG. 1A). Furthermore, despite reducing the generation of both the myeloid (CD33⁺) and lymphoid (CD19⁺) lineages, DLK1 knockdown led to a significant bias towards myeloid differentiation (p-value=0.0372; data not shown). In contrast, DLK1 overexpression, while not affecting overall human engraftment (data not shown), led to a statistically significant increase in CD34⁺ engrafting cells, pointing to delayed differentiation in vivo (FIG. 1B). Taken together, these data suggest that DLK1 regulates HSPC growth and differentiation in vivo.

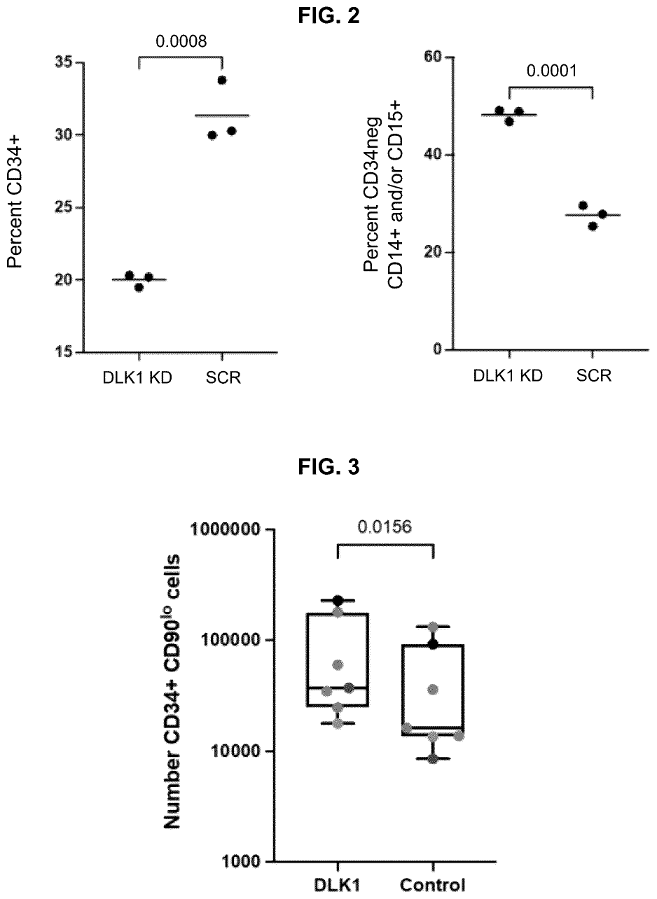

DLK1 knockdown leads to loss of CD34⁺ HSPC along with increased myeloid differentiation during in vitro endothelial cell (EC) co-culture. To directly test whether DLK1 promotes HSPC maintenance and/or self-renewal by delaying differentiation, CB CD34⁺ HSPC were transduced with lentivirus encoding either DLK1 or scrambled (SCR) control shRNAs co-expressing GFP. Resulting cells were subjected to two week co-culture with Akt-activated ECs (henceforth referred to as ECs), an approach previously employed to maintain HSC ex vivo (Butler, et al., Cell Stem Cell, 2010. 6(3): p. 251-64; Walter, et al., Leukemia, 2014. 28(10): p. 1969-77; and Le, et al., Leukemia, 2021. 35(2): p. 601-605), at which time their differentiation was assessed by flow cytometry. As shown in FIG. 2, DLK1 knockdown led to a significant decrease in CD34⁺ percentage (left) and a significant increase in differentiation along the myeloid lineage (right), as characterized by the increased percentage of CD34^{neg} CD14⁺ and/or CD15⁺ cells. These findings further suggest that upon DLK1 knockdown, HSPC more rapidly differentiate with a bias toward the myeloid lineage.

DLK1 overexpression or exposure to exogenous extracellular domain of DLK1 (ECD) leads to an increase in the number of CD34⁺CD90^{lo} HSPC in vitro. Whether DLK1 overexpression altered the in vivo growth and differentiation of less mature precursors was next assessed. To do so, CB CD34⁺ HSPC were transduced with lentivirus encoding either DLK1-GFP or control GFP and cultured in the presence of Stemspan plus 5 growth factors (SS+5GF, wherein the growth factors are SCF, FLT3L, IL-6, and TPO at 50 ng/ml and IL-3 at 10 ng/ml). Relative to starting cells, DLK1 overexpression led to a statistically significant increase in GFP⁺CD34⁺CD90^{lo} HSPC compared to control (FIG. 3). These data suggest that DLK1 overexpression is sufficient to enhance the self-renewal and/or induce a delay in the differentiation of the CD34⁺CD90^{lo} HSPC subset.

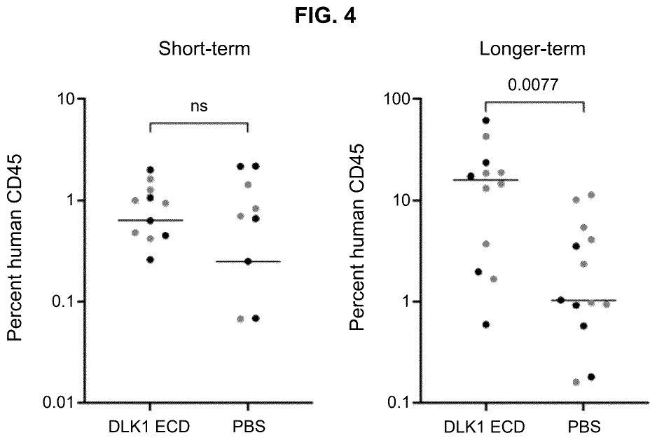

Exposure of CD34⁺ cells to exogenous ECD leads to an increase in longer-term repopulating activity following transplantation. As described above, DLK1 can function in both membrane-bound and soluble (ECD) capacities, the latter of which can act in both an autocrine and paracrine manner. Consequently, whether exogenous ECD addition during ex vivo culture of CB derived CD34⁺ cells could alter repopulating activity following trans-plantation was tested. To do so, CB derived CD34⁺ cells were cultured in the presence of soluble ECD or phosphate buffered saline (PBS) (vehicle control) for four days followed by transplantation into NSG mice. In two independent experiments, ECD did not affect rapid repopulating cells (FIG. 4, left), but led to a significant increase in longer-term repopulating activity (FIG. 4, right). The lack of an effect of ECD on short-term repopulation, compared to the increased two-week engraftment of CD34⁺ human cells upon DLK1 overexpression (FIG. 1B), may be attributed to the temporary exposure of HSPC to ECD in vitro, as opposed to the sustained expression facilitated by integration in vivo.

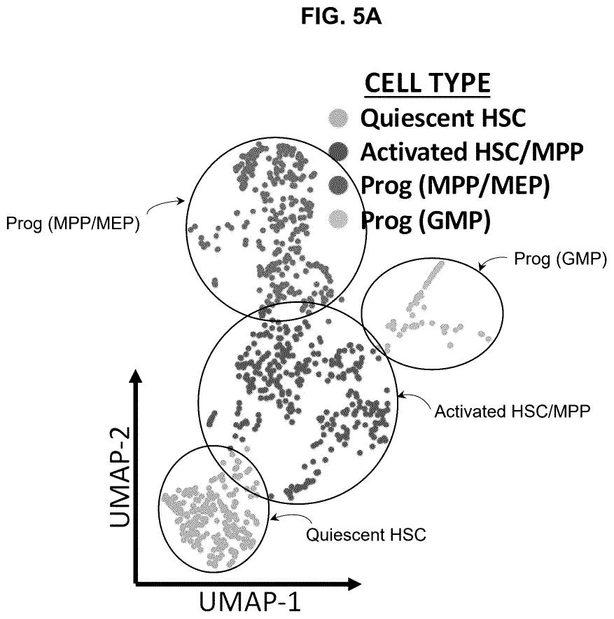

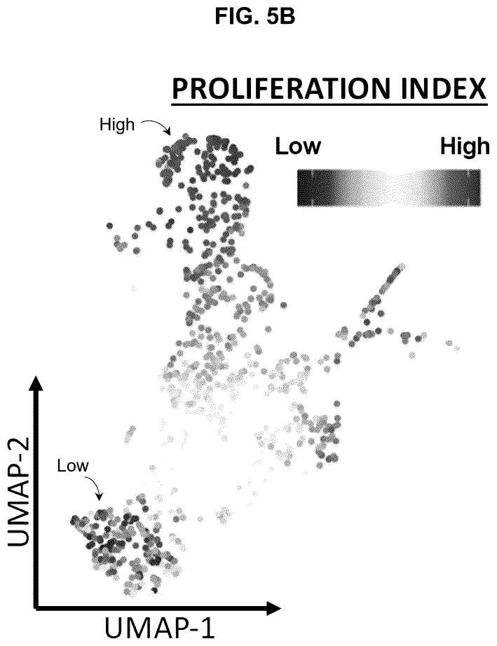

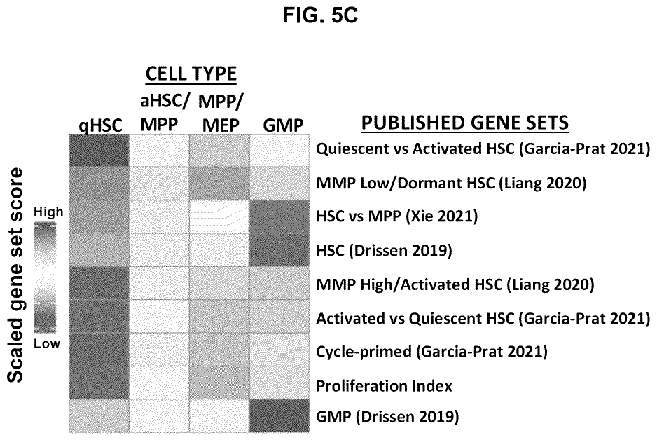

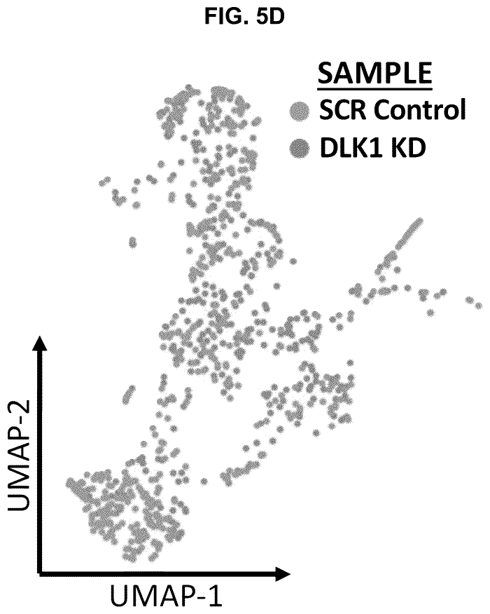

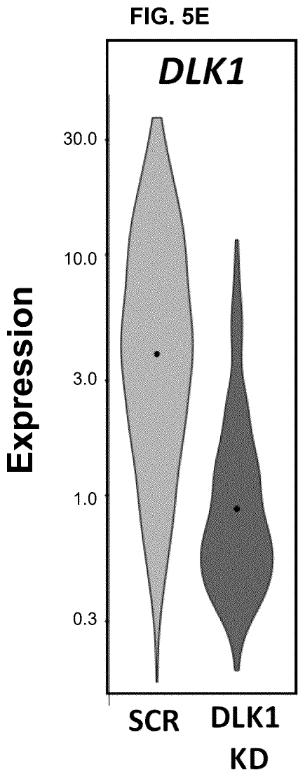

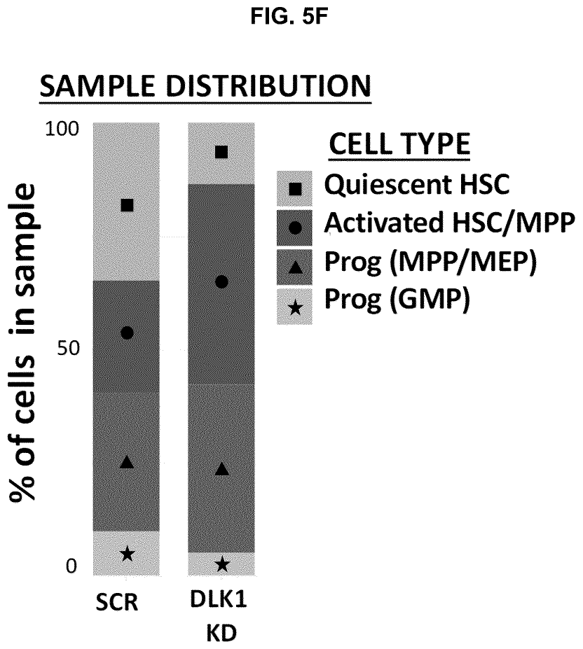

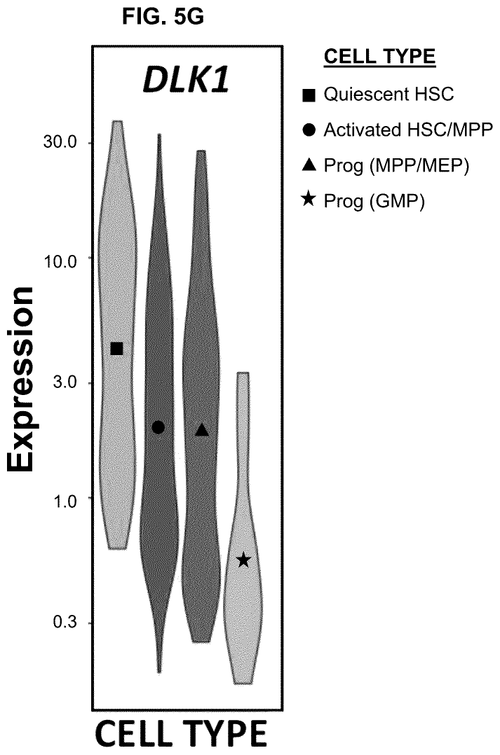

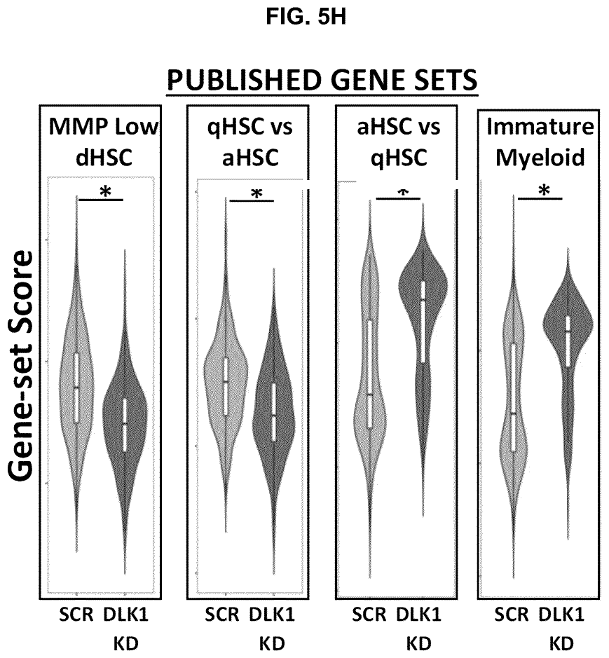

DLK1 knockdown leads to egress of HSC from a transcriptionally quiescent to an activated state. To gain insight into the DLK1-mediated maintenance of HSC, the changes in gene expression that accompany DLK1 knockdown in HSC were next tested. Flow activated cell sorting was used to isolate highly enriched HSC (CD34⁺Lin⁻CD38⁻ CD90^{lo}CD45RA⁻EPCR⁺) from human CB, a subpopulation known to display higher DLK1 expression than more committed progenitors or differentiated cells (Notta, et al., Science, 2011. 333(6039): p. 218-21). Cells were immediately infected with lentivirus expressing DLK1 shRNA or its scrambled control and cultured for 4 days ex vivo. Cells were harvested and subjected to scRNAseq using the 10× Genomics platform. Dimensionality reduction via Uniform Manifold Approximation and Projection (UMAP, McInnes, et al., ArXiv, 2018. abs/1802.03426) in combination with Louvain clustering (Blondel, et al., Journal of Statistical Mechanics—Theory and Experiment, 2008) and supervised cell type classification using canonical marker genes and published gene sets (Liang 2020; Garcia-Prat, et al., Cell Stem Cell, 2021. 28(10): p. 1838-1850 e10; and Drissen, et al., Sci Immunol, 2019. 4(35)) identified a quiescent HSC (qHSC) subset, a non-quiescent, cycling activated HSC (aHSC) or multipotent progenitor (MPP) subset, and progenitors starting to differentiate along the megakaryocyte/erythroid (MEP) or granulocyte/monocyte (GMP) lineages (FIGS. 5A-5C). Notably, compared to scrambled control cells (SCR), DLK1 knockdown led to a statistically significant decrease in the proportion of qHSC and a corresponding increase in the proportion of aHSC/MPP (Chi-squared p value=1.48e-14). Furthermore, compared to control (SCR), DLK1 knockdown resulted in a significant reduction in gene set scores from published gene signatures associated with metabolically dormant (i.e., low mitochondrial membrane potential (low MMP)) or quiescent HSC state along with a corresponding increase in gene set scores associated with an activated/cycling HSC state and early/immature myeloid differentiation (Liang 2020; Velten 2017; and Garcia-Prat, et al., Cell Stem Cell, 2021. 28(10): p. 1838-1850 e10) (FIGS. 5D-5H). These results, which have now been confirmed in a second experiment (data not shown), suggest that DLK1 promotes the maintenance of HSC quiescence.

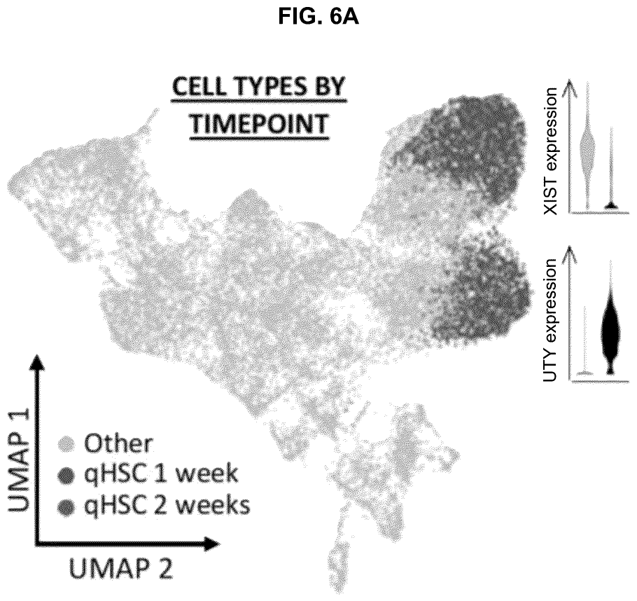

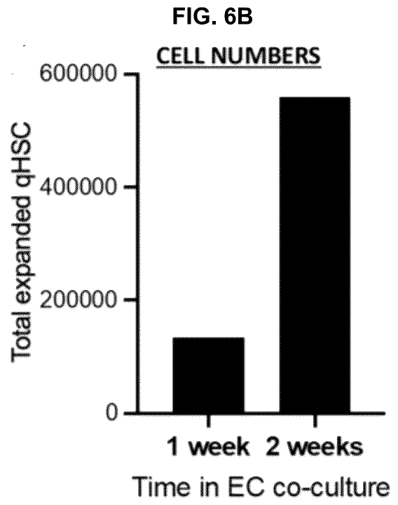

EC co-culture of enriched HSC allows for both the maintenance and expansion of qHSC. The suggestion that DLK1 maintains or expands the quiescent stem cell pool compels the need for an in vitro model with which to assess DLK1 effects on HSC quiescence. The use of EC co-culture was explored, which has previously been demonstrated to expand long-term repopulating murine HSC (Butler, et al., Cell Stem Cell, 2010. 6(3): p. 251-64) and CB repopulating cells (Butler, et al., Blood, 2012. 120(6): p. 1344-7). CB-derived highly enriched HSC were cultured on ECs for one or two weeks, at which time cells were harvested and subjected to scRNAseq. The Harmony algorithm (Korsunsky, et al., Nat Methods, 2019. 16(12): p. 1289-1296) in Seurat (Butler, et al., Nat Biotechnol, 2018. 36(5): p. 411-420) was used to project cells into a shared UMAP embedding. Two qHSC clusters (FIG. 6A) were identified, as previously defined by supervised cell type classification using canonical marker genes and published gene sets (FIG. 5). Differential gene expression analysis determined that the two qHSC clusters separated due to differences in sex-specific gene expression (FIG. 6A, right). The potential impact of sex bias on gene expression was monitored, as sex-related genes have been shown to influence HSC growth and differentiation (Yang, et al., Nat Commun, 2022. 13(1): p. 4464; and Cui, et al., Blood Sci, 2022. 4(4): p. 185-191). Upon relating the proportion of qHSC to total cells generated during culture, a 4.2-fold increase was observed in qHSC numbers between one and two weeks of EC co-culture (FIG. 6B). These data suggest that EC co-culture system not only maintains qHSC during division but may also induce aHSCs to return to quiescence after division.

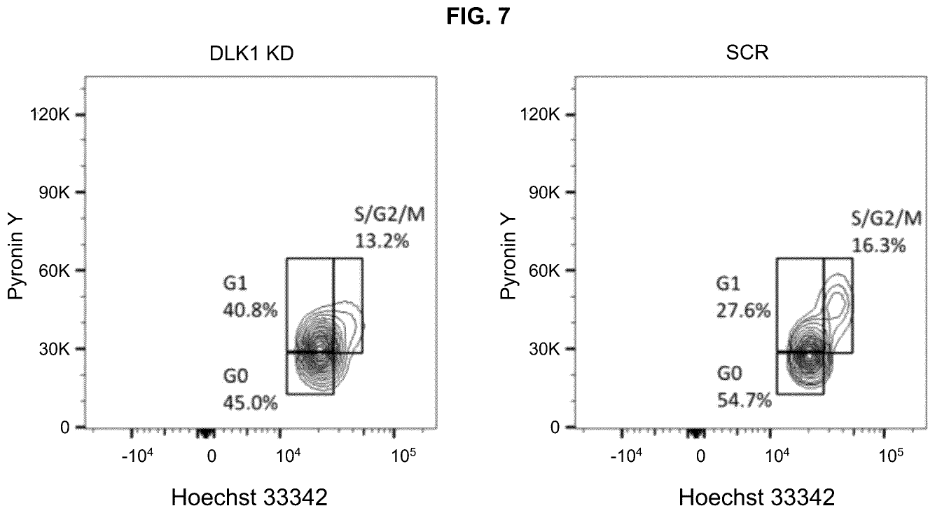

DLK1 knockdown leads to loss of qHSC/G0 cells during EC co-culture. A portion of the enriched HSC used in scRNAseq experiment (FIG. 6) were independently subjected to DLK1 knockdown and one week of EC co-culture, at which time quiescent state was assessed by flow cytometry using Pyronin Y and Hoechst 33342 staining. DLK1 knockdown led to a 9.7% difference in G0 cells, representing an 18% reduction compared to control (SCR) cells (FIG. 7). These results suggests that DLK1 maintains and/or expands qHSC by inducing aHSC to return to quiescence following division.

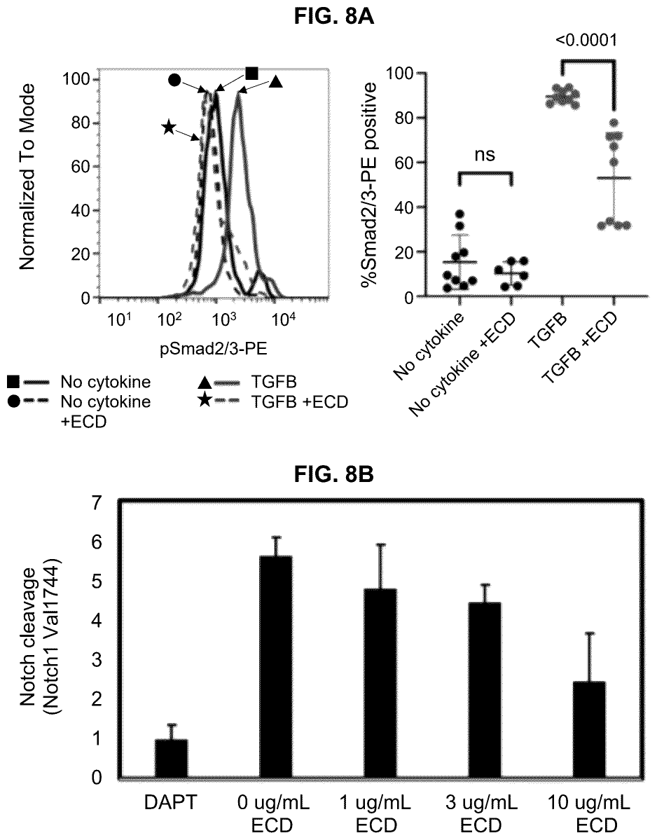

DLK1 Mechanism of action. ECD inhibits multiple signaling pathways. Immunoprecipitation was used followed by mass spectrometry to identify proteins that reproducibly co-immunoprecipitate with DLK1 from membrane solubilized KG1a cells (data not shown). Several receptor signaling pathways known to regulate HSPC (Traustadottir, et al., Cytokine Growth Factor Rev, 2019. 46: p. 17-27) were found. ECD inhibition of two of these pathways, TGFβ and Notch, were initially tested. To assess changes in TGFβ signaling, the level of SMAD2/3 phosphorylation (pSMAD2/3) after stimulation with TGFβ ligand was measured (FIG. 8A). To assess changes in Notch signaling, the level of receptor cleavage during co-cultivation of cells with immobilized Notch ligand was measured (FIG. 8B). Importantly, the proteomics study suggested that ECD interacts with Presenilin-1 (PSEN1) and Nicastrin, components of the gamma secretase enzyme complex that cleaves several receptors, including Notch. Notch was non-significantly pulled-down by DLK1 and DLK1 is not a substrate of gamma secretase as its cleavage pattern was not impacted by gamma secretase inhibition with DAPT (data not shown).

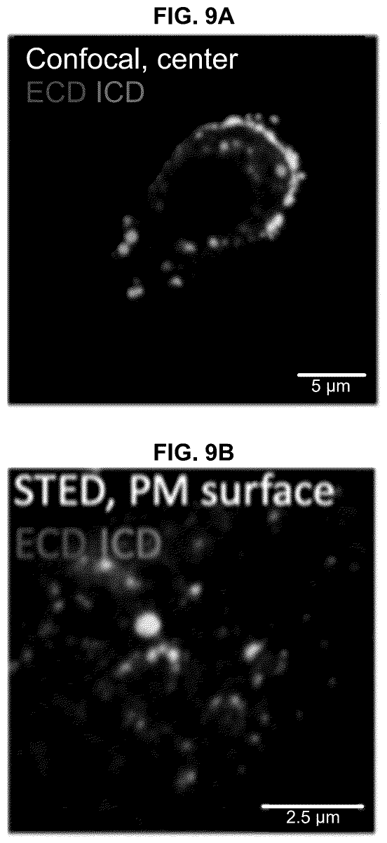

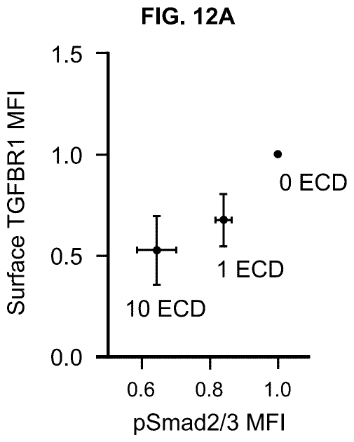

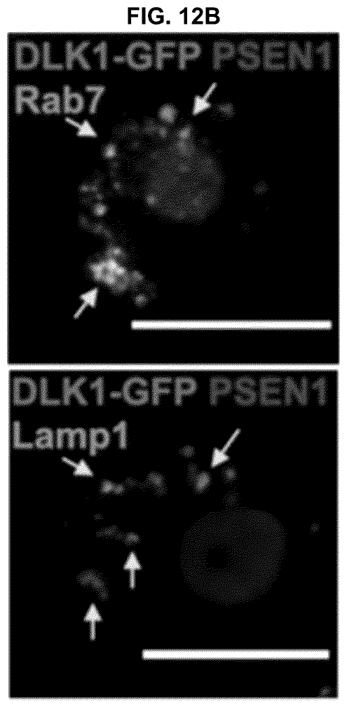

DLK1 plasma membrane organization informs on its function. To observe DLK1 organization, KG1a cells stably expressing DLK1 as a C-terminal GFP fusion protein (DLK1-GFP) were stained with an antibody specific to the ECD. Using confocal microscopy, DLK1 was found to be enriched in the plasma membrane (PM) as well as intracellularly (FIG. 9A). Intracellular DLK1 localizes to early-late endosomes and lysosomes, determined using immunofluorescence (e.g., FIG. 12B). Subsequent use of stimulated emission depletion (STED) microscopy revealed that DLK1 in the PM is not uniform but organized into particles that resemble phase separated condensates (FIG. 9B). Using FIJI analysis, the number, size, and intensity of ECD and GFP signals within condensates were determined (data not shown). Consistent with the findings that ECD is sufficient to increase longer-term repopulating activity (FIG. 4) and inhibit TGFβ signaling and Notch cleavage (FIG. 8), cleaved ECD (ECD+/GFP) represents 75% of the total DLK1 condensates, suggesting ECD is sufficient to phase separate and form condensates.

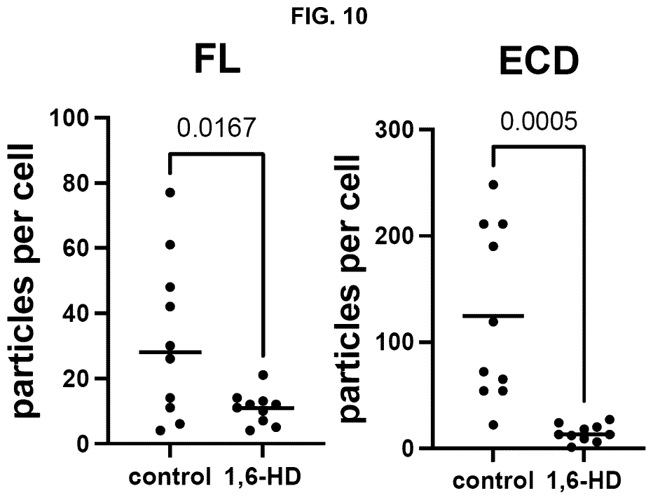

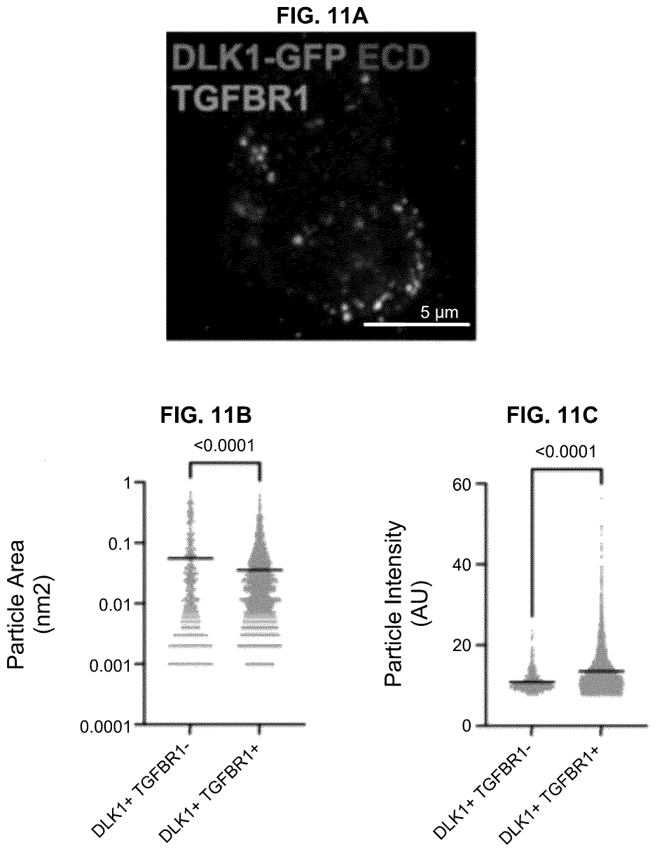

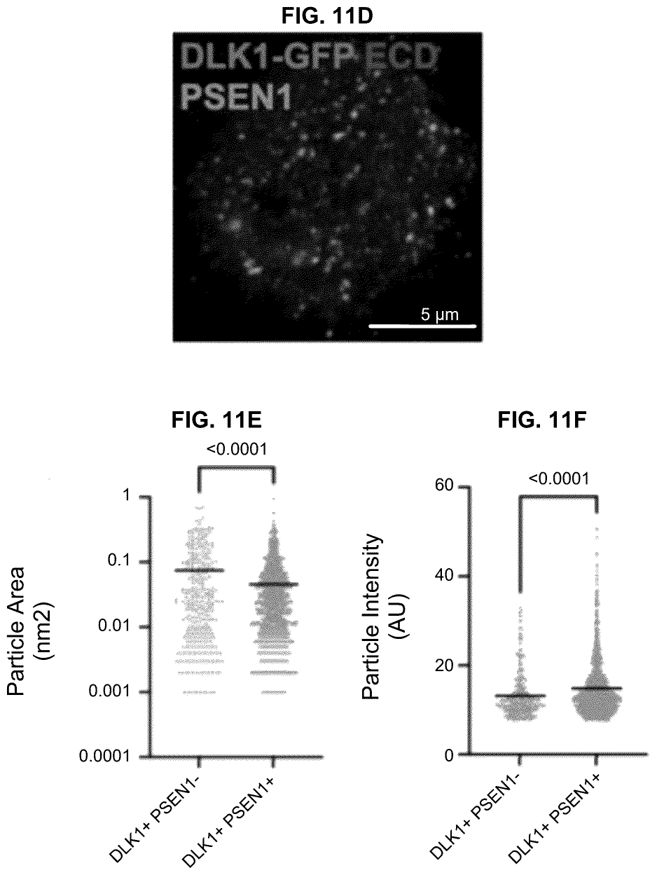

DLK1 phase separates and co-localizes with receptor components. DLK1 condensates, especially ECD condensates, are sensitive to 1,6-HD (FIG. 10). Using STED microscopy to image DLK1 condensates within the PM of KG1a-DLK1-GFP cells (30 nm resolution), co-localization of DLK1 with TGFβR1 was observed (FIG. 11A). Quantitative analysis of 3691 condensates over 10 images reveals that 75% of DLK1 in the PM overlaps with TGFβR1 and 75% of TGFβR1 overlaps with DLK1 (data not shown). Similar results were obtained for the co-localization of DLK1 with PSEN1 (FIG. 11D), where analysis of 1864 condensates over 10 images reveals that 80% of DLK1 in the PM overlaps with PSEN1 and 75% of PSEN1 overlaps with DLK1 (data not shown). The overlap of DLK1 with TGFβR1 (75%) and PSEN1 (80%) suggests that TGFβR1 and PSEN1 are likely in the same DLK1 condensates.

Whether DLK1 condensate size or intensity differed among DLK1⁺Receptor⁺ and DLK1⁺Receptor condensates, with use of ‘Receptor’ representing both TGFβR1 and/or the receptor component PSEN1, was tested. Despite having a smaller area (FIGS. 11B, 11E), ECD intensity was significantly higher in condensates that overlap with TGFβR1 or PSEN1 suggesting that DLK1 concentration increases in receptor positive condensates, consistent with a PS mechanism (FIG. 11C, 11F, respectively).

The effect of the ECD on receptor dynamics was further investigated and it was observed that surface levels of TGFβR1 decrease in a dose-dependent manner after ECD addition, in part explaining the ECD-dependent decrease in pSmad2/3 levels (FIG. 12A). Notably, this effect did not happen after a 30 minute ECD addition (data not shown) but was robust after longer timepoints. These data suggest that DLK1 does not inhibit signaling immediately but may act through modulating receptor recycling and/or turnover, thereby preventing either TGFβR restimulation by TGFβ ligand or gamma secretase binding to additional Notch receptors. DLK1 co-localization was observed with TGFβR1 and PSEN1 in endosomes and lysosomes (data for PSEN1 shown in FIG. 12B).

Prophetic Example 1. Determine whether DLK1 maintains or induces a quiescent state to prevent HSC proliferation and differentiation. Determine the effect of DLK1 knockdown on qHSC dynamics ex vivo and in vivo. The effects of DLK1 knockdown on maintaining qHSC and/or inducing the return of aHSC to a quiescent state following EC co-culture will be determined, assessing both the generation of transcriptionally-defined qHSC and functional longer-term repopulating HSC. To do so, two distinct strategies to isolate qHSC or aHSC subsets will be employed prior to or following culture of highly enriched HSC (CD34⁺ Lin⁻ CD38⁻ CD90^{lo} CD45RA⁻ EPCR⁺), isolated from CB by fluorescence activated cell sorting (FACS): 1) Pyronin Y/Hoechst staining to isolate cells based on cell cycle status; 2) CSFE staining to isolate HSC based on divisional history. Details of each experimental approach are described below, along with methods to assess quiescence by scRNAseq and HSC function by transplantation.

Isolation of non-cycling vs. cycling HSC subsets via Pyronin Y and Hoechst staining. Highly enriched HSC will be FACS-separated into enriched qHSC and aHSC subpopulations based on their cell cycle status using Pyronin Y and Hoechst 33342 staining. Specifically, Pyronin Y^{lo} and Hoechst 33342^{lo} HSC represent non-cycling (G0) qHSC, while Pyronin Y^{hi} and Hoechst 33342^{lo} represent their cycling (G1) aHSC counterparts. However, these G0 qHSC may not represent the most deeply quiescent subset. As such, whether tetramethylrhodamine ethyl ester (TMRE) allows further subsetting of these G0 cells based on mitochondrial activity (Liang 2020; Vannini, et al., Nat Commun, 2016. 7: p. 13125; and Sukumar, et al., Cell Metab, 2016. 23(1): p. 63-76) will be assessed, a possibility suggested by previous identification of a DLK1-dependent TMRE^{lo} subset following EC co-culture (data not shown). If this is the case, subsequent experiments will incorporate the use of TMRE to isolate the most deeply quiescent HSC. Enriched qHSC and aHSC subsets will be isolated 1) prior to and 2) following culture for use as described below:

Prior to culture, the isolated enriched qHSC and aHSC subsets will be transduced with lentivirus encoding DLK1 or scrambled control shRNAs co-expressing GFP. Transduced cells will be placed into culture on ECs (Butler, et al., Blood, 2012. 120(6): p. 1344-7) in the presence of Stemspan plus 3 growth factors (SCF, FLT3L, TPO at 50 ng/ml). Following one and two weeks of EC co-culture, cells will be harvested for (i.) scRNAseq or (ii.) transplantation into immune deficient mice. Following culture, a portion of harvested cells will also be separated into their qHSC and aHSC subsets using the Pyronin Y/Hoechst isolation method, potentially in combination with TMRE staining (Liang 2020). Isolated subsets will be subjected to scRNAseq and in vivo repopulation assays. This latter approach will facilitate a robust assessment of qHSC and aHSC subsets generated during EC co-culture, relating their flow-based phenotypic characteristics to their transcriptionally-defined quiescent state (scRNAseq) and functional properties (transplantation).

Isolation of non-divided vs. divided HSC subsets via CSFE staining. Highly enriched HSC will be labeled with CSFE to follow divisional history and transduced with lentivirus encoding DLK1 shRNA or its scrambled control co-expressing mCherry. Transduced cells will be transferred to ECs, where cultured cells will be assessed by flow cytometry to identify a time point(s) whereby half of the cells remain undivided (100% CSFE) while the remainder have completed one to two (50%-25% CSFE) cell divisions. These non-divided and divided subpopulations will be sorted and placed back into culture on ECs for a total culture period of one to two weeks, at which time cells will be harvested from each group and, as described above, subjected to (i.) scRNAseq or (ii.) transplantation into immune deficient mice.

Methods for assessment of changes in gene expression/cellular state by scRNAseq.

Briefly, scRNAseq of harvested qHSC and aHSC will be performed using methods as described in Experimental Example 1, assessing three independent biological replicates to ensure scientific rigor. Different here, qHSC and aHSC from DLK1 knockdown or scrambled control groups will be pre-incubated with TotalSeq cell hashing antibodies (Biolegend), as previously done in (Ishida, et al., Differentiation latency and dormancy signatures define fetal liver HSCs at single cell resolution. bioRxiv, 2023 (“Ishida 2023”)), such that each sample can be processed simultaneously in a single lane (Stoeckius, et al., Genome Biol, 2018. 19(1): p. 224). Gene and hash antibody-specific oligo barcode expression matrices for single cells will be generated from raw sequencing data using the Cell Ranger pipeline (10× Genomics). Using established computational pipelines (Ishida 2023; Dignum, et al., Cell Rep, 2021. 36(11): p. 109675; and Hadland, et al., Nat Commun, 2022. 13(1): p. 1584), scRNAseq data will be mined to deduce whether the quiescent population observed following two weeks of EC co-culture is derived primarily from qHSC or aHSC subpopulations and whether this outcome is altered following perturbation of DLK1 expression. Briefly, Monocle 3 (Cao, et al., Nature, 2019. 566(7745): p. 496-502) and Seurat (Liu, X., et al., Genome Biol, 2021. 22(1): p. 230) scRNAseq analysis packages will be used to: 1) perform quality control steps including removal of doublets and poor quality cells, normalization of reads, and sample alignment; 2) assign hashing oligos to each condition tested; 3) deduce cell types based on semi-supervised cell type classification algorithms (Pliner, et al., Nat Methods, 2019. 16(10): p. 983-986) using established marker genes; 4) compare normalized gene-set scores of relevant published and annotated gene sets of HSC states across timepoints and conditions, including those indicative of quiescence, cell cycle entry (including MK167 expression), myeloid biased differentiation and metabolic status (Liang 2020; Velten 2017; Garcia-Prat, et al., Cell Stem Cell, 2021. 28(10): p. 1838-1850 e10; Drissen, et al., Sci Immunol, 2019. 4(35); Xie, et al., Blood Cancer Discov, 2021. 2(1): p. 32-53; Kaufmann, et al., Nat Immunol, 2021. 22(6): p. 723-734; and Roy, et al., Cell Rep, 2021. 36(11): p. 109698) and 5) perform advanced cell cycle analysis (Riba, et al., Nat Commun, 2022. 13(1): p. 2865; and Scialdone, et al., Methods, 2015. 85: p. 54-61).

Methods for assessment of longer-term repopulating activity by transplantation in immune deficient mice. It has been shown that qHSCs are responsible for long-term repopulation (Passegue, et al., J Exp Med, 2005. 202(11): p. 1599-611). Relevant here, it has been shown that human qHSC have increased longer-term repopulating activity in immune deficient mice compared to their cycling counterparts (Qiu, et al., Blood Adv, 2021. 5(6): p. 1605-1616). Furthermore, murine aHSC, isolated via TMRE staining, represented less than 1% of longer-term (16 week) repopulating cells, compared to their qHSC counterparts (Liang 2020) following primary transplantation. Here, whether transcriptionally-defined qHSC, derived from aHSC's return to quiescence during EC co-culture, acquired robust longer-term repopulating activity in a DLK1-dependent manner will be determined. The progeny of the same number of starting cells will be transplanted into both male and female immune deficient NSG mice of the same age and weight, assessing primary engraftment 4-6 months post-injection. Long-term self-renewal potential following serial transplantation will also be assessed. Statistically significant differences in human engraftment between DLK1 knockdown and control groups will be determined using the t-test. Moreover, long-term repopulating cells will be enumerated by limit-dilution transplantation, with the frequency of multilineage repopulating cells determined following flow cytometric analysis using L-calc (Stem Cell Technologies). To ensure scientific rigor, a power calculation will be used to determine the minimal number of mice necessary to make statistically meaningful interpretations for each experiment (see Vertebrate Animals section). qHSC will also be enumerated by limit dilution transplant of enriched HSC prior to initiation of culture. If necessary, the effectiveness of cell numbers will be enhanced by using intrafemoral injections and the established colony of humanized NSG-SGM3 mice as recipients (Wunderlich, et al., Leukemia, 2010. 24(10): p. 1785-8).

Determine whether exposure to ECD during EC co-culture alters qHSC dynamics. Whether exposure of HSC to exogenous ECD enhances repopulation by maintaining qHSC or returning aHSC to a quiescent state will also be tested. The same qHSC/aHSC isolation approaches as described above will be used. Isolated qHSC and aHSC subsets will be placed into culture on ECs with increasing concentrations of mammalian recombinant ECD (R&D) or, to ensure uniform ECD exposure over time, DLK1-expressing ECs. As a control, isolated subsets will also be exposed to ECD during short term culture, as described in Experimental Example 1. Following culture, cells will be harvested and effects of ECD exposure determined using (i.) scRNAseq or (ii.) transplantation into immune deficient mice.

DLK1 delays stem cell differentiation by maintaining qHSC and inducing aHSC to return to quiescence. As such, in the context of DLK1 knockdown, scRNAseq data will reveal decreased numbers of transcriptionally-defined qHSC originating from aHSC compared to control. Consequently, DLK1 knockdown will lead to diminished multi-lineage repopulation, as suggested by murine studies (Huang, et al., Exp Hematol Oncol, 2023. 12(1): p. 11). In contrast, exposure to exogenous or EC-derived ECD will increase repopulation frequency due to expansion of qHSC, largely derived from aHSC returning to quiescence. Such findings would lay the groundwork for future studies to explore the use of exogenous ECD to maintain or expand longer-term HSC capacity ex vivo for therapeutic purposes, including HSC-based gene therapy.

### Prophetic Example 2. Determine the Mechanism of DLK1 Inhibition of Receptor Signaling within Condensates

Characterizing ECD concentration and homo-oligomerization in PM condensates. To test if ECD intensity is higher in receptor positive condensates because of ECD oligomerization, single molecule microscopy techniques will be utilized. Specifically, DNA point accumulation will be utilized for imaging in nanoscale topography (DNA-PAINT) to first define the concentration of ECD within receptor positive and negative condensates. DNA-PAINT uses short fluorophore-conjugated imaging strand (IS) oligonucleotides that transiently bind to a complementary docking strand (DS) oligonucleotide attached to the target of interest to yield short-lived ‘blinking’ events. These events are then localized with sub-diffractive precision (8-10 nm), reporting the position of target molecules. These data will be used to calculate ECD PM concentrations and estimate ECD monomer and oligomer sizes (Nan, et al., Proc Natl Acad Sci USA, 2015. 112(26): p. 7996-8001; Jungmann, et al., Nat Methods, 2016. 13(5): p. 439-42; and Xu, et al., J Phys Chem Lett, 2019. 10(19): p. 5836-5840). This will show that ECD is oligomerized and discern the extent of DLK1 oligomerization within receptor positive and negative condensates. Next, the properties of ECD condensates will be further characterized using fluorescence recovery after photobleaching (FRAP). FRAP can not only inform on protein properties, such as diffusion of ECD in and out of condensates, but also their function. Preliminary FRAP experiments (not shown here) demonstrate that ECD condensates resemble an inhibitory condensate (slow diffusion), in agreement with the Experimental Example 1 results demonstrating inhibition of TGFβ signaling and Notch receptor cleavage (FIG. 8). Next, chemical perturbations will be used to screen for the intramolecular interactions that drive ECD condensate formation. ECD phase separates in part through hydrophobic interactions (FIG. 10). However, most PS events occur through multivalent interactions, therefore perturbation of electrostatics (using salt) and disulfide formation (using CoCl₂/DFO/DTT/NAC) will be performed (Reed, E. H. and D. A. Hammer, Soft Matter, 2018. 14(31): p. 6506-6513; and Vernon, et al., Elife, 2018. 7). Using STED and DNA-PAINT, changes in ECD condensates will be quantitated after treatments. Investigation of ECD cysteine residues (36 cysteines, 13% of ECD) is of particular interest as manipulating the redox state in TGFβ signaling assays can enhance ECD potency (data not shown). Successful completion of the proposed experiments will result in a deeper understanding of the molecular organization of the ECD within condensates and the potential for chemical manipulation of ECD condensate formation (Mitrea, et al., Nat Rev Drug Discov, 2022. 21(11): p. 841-862).

Characterizing DLK1 interaction with PM receptor complexes and the consequence of ECD on receptor dynamics. To study receptor dynamics, DNA-PAINT will be used in combination with foster resonance energy transfer-coupled fluorescence lifetime imaging microscopy (FRET/FLIM). In live or fixed cells, FRET is capable of resolving molecular interactions and conformations with a spatial resolution of 3-6 nm. FRET/FLIM is advantageous compared with conventional FRET as it is not susceptible to variations in fluorophore concentration (Bajar, et al., Sensors (Basel), 2016. 16(9)). Incorporation of non-canonical amino acids to the extracellular domains of DLK1 and receptors of interest will allow for efficient labeling on live cells with click chemistry (Kuhlemann, et al., Front Synaptic Neurosci, 2021. 13: p. 727406; and Koester, et al., Curr Protoc, 2022. 2(11): p. e618). These studies will characterize DLK1 interactions with receptors of interest within condensates at unprecedented detail. For TGFβ interactions, this would include determining whether DLK1 interacts with receptors prior to ligand binding or alters the formation of ligand bound-tetrameric TGFβ receptor assembly necessary for signal propagation. For Notch related interactions, this would include determining whether DLK1 alters the assembly of the gamma secretase complex or its proximity to Notch. Using the methodologies described above, changes in the trafficking and/or turnover of receptor components with ECD will also be measured. To do so, the distribution of TGFBRs and PSEN1 will be examined in endosomes and lysosomes pre- and post-exogenous ECD exposure. An ECD-dependent increase in the lysosomal distribution of receptor components would suggest increased turnover due to lysosomal degradation. Additionally, receptor turnover in the presence or absence of ECD will be studied using pulse-chase experiments (such as with 35S methionine and/or biotinylation of surface receptors), measuring protein levels by immunoblotting against TGFβRs and gamma secretase components.

For each new tag system generated, quality control signaling assays will be performed to ensure normal function (lacking significant difference from control system), including TGFβ signaling and Notch cleavage assays. Alternate tagging systems such as with non-canonical amino acids and click chemistry could also be used.

Shotgun scanning mutagenesis can also be used, an alternative approach that circumvents the need to purify protein or perform biophysical characterization while screening mutants. Shotgun scanning mutagenesis combines binomial mutagenesis properties with phage display technology to mutagenize up to 20 residues to alanine simultaneously (Weiss, et al., Proc Natl Acad Sci USA, 2000. 97(16): p. 8950-4; and Bazan, et al., Hum Vaccin Immunother, 2012. 8(12): p. 1817-28). ECD presenting phages will be screened for mutants that retain the ability to interact with cells expressing either WT ECD or receptor components (individually or all) but not both. Sequence analysis will determine the ratio of WT/alanine at each residue which will determine hotspot regions within ECD that are involved in PS. As an alternate approach to the use of hypoxia mimetic agents, cells can be grown directly under conditions of low oxygen.

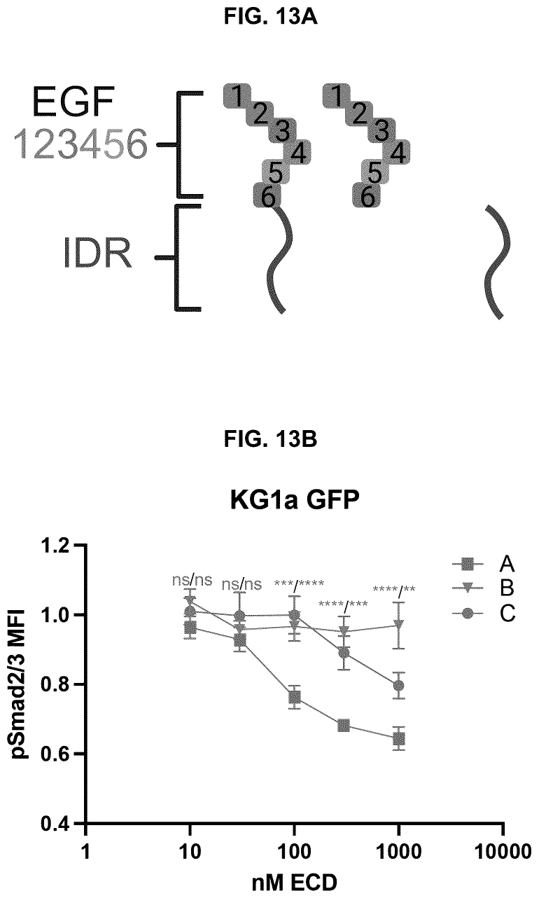

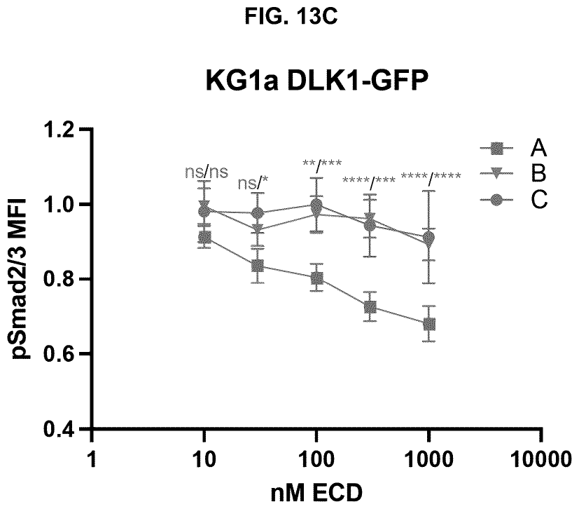

Experimental Example 2. Generate ECD mutants to identify protein features that drive ECD interactions within condensates. To further understand the mechanism of ECD PS with receptor components, ECD mutants were generated to determine whether distinct ECD domains are responsible for homotypic and/or heterotypic interactions. PS occurs through multivalent interactions between repeating protein domains, intrinsically disordered regions, post-translational modifications, cysteine residues, hydrophobic residues, and polar residues. The ECD contains 8 domains consisting of an intrinsically disordered signal peptide (SP), 6 EGF repeats, and an intrinsically disordered domain (IDR) (FIG. 13A). A previous report suggested the ECD IDR is involved in homotypic oligomerization (Baladron, et al., Front Biosci, 2001. 6: p. A25-32), while heterotypic interactions were reported to occur between DLK1 EGF domains and cysteine rich growth modulating molecules (Baladron, et al., Biochem Biophys Res Commun, 2002. 291(2): p. 193-204), the latter of which suggests that inter- and intradomain disulfides may contribute to DLK1 PS with receptor components. With these previous reports in mind, two initial ECD mutant and WT proteins were produced using lentiviral-based expression in mammalian Freestyle 293F cells. ECD proteins were purified via a His-tag and assayed for activity using ac TGFβ signaling assay (FIG. 8). As a control, ECD-His (A) was used (FIG. 13B, 13C). Notably, initial two mutants support that both the EGF and IDR region of the protein are essential for inhibition of TGFβ signaling (FIG. 13B, 13C).

### Prophetic Example 3. Determine Whether Inhibition of Multiple Receptor Signaling Pathways is Required for the Effects of DLK1 on the Maintenance and/or Induction of HSC Quiescence

It is expected that the combined effects of fine tuning multiple signaling pathways is necessary for DLK1 to maintain/induce the qHSC state and that perturbing individual pathways will not reverse the full effects of DLK1 knockdown on qHSC dynamics. To test this, it will be determined whether the individual or combined perturbation of TGFβ and Notch pathways is sufficient to reverse the effects of DLK1 knockdown or ECD exposure. In support of the need to suppress the TGFβ pathway to maintain HSC quiescence, lower physiologic doses of TGFβ ligand have been shown to drive the proliferation of myeloid-biased HSC (Challen, et al., Cell Stem Cell, 2010. 6(3): p. 265-78). With respect to Notch, while a recent report suggests that Notch signaling activation is involved in the maintenance of murine long-term repopulating HSC in vivo (Huang, et al., Exp Hematol Oncol, 2023. 12(1): p. 11), numerous studies have shown that long-term repopulating HSC are Notch signaling independent (Maillard, et al., Cell Stem Cell, 2008. 2(4): p. 356-66; and Varnum-Finney, et al., J Clin Invest, 2011. 121(3): p. 1207-16). Moreover, non-cleaved Notch receptor can act as an adhesion molecule, allowing HSC to bind to stromal cells, preventing their cycling and egress from the niche (Wang, et al., Stem Cells, 2015. 33(7): p. 2280-93). Signaling networks beyond TGFβ and Notch that are known to affect HSC and are inhibited by DLK1 will also be identified.

Methods. Determine whether modulation of TGFβ signaling and/or Notch receptor cleavage can reverse the effect of DLK1 knockdown or ECD exposure on qHSC dynamics ex vivo and in vivo. It will be determined whether perturbation of receptor signaling pathways individually and in combination are sufficient to reverse the effects of DLK1 knockdown or ECD exposure on maintaining qHSC and/or inducing the return of aHSC to a quiescent state.

DLK1 knockdown: Enriched HSC and/or more refined qHSC and aHSC subsets transduced to express DLK1 shRNA or its scrambled control will be placed into culture on ECs. To quantitively reduce TGFβ signaling and/or Notch cleavage, media will be supplemented with varying concentrations of 1) anti-TGFβ antibody (clone 1D11, Thermo Fisher, Waltham, MA) or its isotype control; 2) anti-Notch1 and/or Notch2 receptor antibodies that inhibit Notch cleavage and activation (Le, et al., Leukemia, 2021. 35(2): p. 601-605; and Wu, et al., Nature, 2010. 464(7291): p. 1052-7), or their isotype control; or 3) a combination of anti-TGFβ antibody and anti-Notch inhibitory antibodies.

ECD exposure: As an alternative to DLK1 knockdown, activation of TGFβ and/or Notch receptors can be assessed for whether they can reverse the ability of exogenous ECD to induce the quiescence of activated HSC, where the effects of ECD are titratable. In this case, enriched HSC and/or qHSC and aHSC subsets will be cultured on ECs with increasing concentrations of mammalian recombinant ECD (R&D) in the presence and absence of varying concentrations of 1) TGFβ ligand or its vehicle control; 2) engineered Notch ligands bound to beads or beads alone; or 3) a combination of TGFβ and immobilized Notch ligands. In contrast to exposure to soluble Notch ligands, immobilized Notch ligands are activating (Varnum-Finney, et al., Blood, 1998. 91(11): p. 4084-91 (“Varnum-Finney 1998”); and Varnum-Finney, et al., J Cell Sci, 2000. 113 Pt 23: p. 4313-8). In the context of EC co-culture, Notch ligands will be immobilized on beads (Varnum-Finney 1998).

Following one to two weeks of EC co-culture, cells will be subjected to (i) scRNAseq and (ii) transplantation to determine whether perturbation of receptor signaling pathways individually and in combination reversed the effects of DLK1 knockdown or ECD exposure on qHSC.

Determine whether DLK1-mediated inhibition of other receptor signaling pathways contributes to maintaining/promoting HSC quiescence. A list of several signaling receptors known to regulate HSC were compared with scRNAseq data detailing the expression of receptors and ligands in isolated enriched HSC following a short four day culture period. Together the datasets converged on three pathways: TGFβ (TGFβR1, TGFβR2, ENG, ACVR1B), those cleaved by the gamma secretase complex (TNFR1, CD44, Notch), and Integrins (ITGA2B, ITGB2, ITGAL). It will be assessed whether the TNF signaling pathway is also inhibited by DLK1 to facilitate HSC quiescence. TNF-α stimulation of TNFR1 in vivo promotes cell cycle activation with a shift to metabolically activated MPP and a myeloid primed state, resulting in a temporary engraftment defect of TNF-α treated HSC associated with exit from quiescence (Yamashita, et al., Cell Stem Cell, 2019. 25(3): p. 357-372 e7; He, et al., Blood, 2020. 136(2): p. 183-198; Grants, et al., Blood, 2020. 135(25): p. 2235-2251; Etzrodt, et al., Blood, 2019. 133(8): p. 816-819; and Pietras, et al., Blood, 2017. 130(15): p. 1693-1698). DLK1 is expected to have a role in attenuating pro-inflammatory TNF signaling through inhibition of TNFR1 to sustain HSC quiescence. Using a commercially available TNF reporter cell line (InvivoGen, San Diego, CA), it will be assessed whether ECD addition modifies TNFR signaling after stimulation with TNF-α and/or blocks production of soluble TNFR1 intracellular domain after PMA stimulation. Guided by the results, microscopy techniques will be used to further characterize DLK1-TNFR1 interactions. Once DLK1 inhibition of the TNF pathway has been shown, its relevance to DLK1 effects on qHSC dynamics using signaling perturbation approaches to rescue effects of DLK1 knockdown/ECD exposure on inducing the return of aHSC to a quiescent state will be assessed.

Given reports that DLK1 inhibits adipocyte differentiation through integrin a5b1 signaling (Cheng, et al., Eur J Pharmacol, 2021. 909: p. 174385) the potential effect(s) of DLK1-integrin interactions on HSC quiescence will also be examined.

Perturbation of TGFβ signaling and/or Notch receptor cleavage will be unable to fully rescue effects of DLK1 knockdown on qHSC dynamics. However, it is expected that a partial rescue will occur. A partial rescue would indicate that DLK1, presumably by interacting with TGFβR1/2 and/or members of the gamma secretase complex within condensates, inhibits TGFβ signaling and Notch receptor cleavage, respectively, within HSC and suggest that other receptor signaling pathways contribute to DLK1 effects in maintaining qHSC. As such, it is also expected to show that DLK1 interacts with TNF signaling pathway components, inhibiting the TNF signaling in vitro, and that TNF pathway perturbation partially rescues the effects of DLK1 knockdown on qHSC dynamics.

(ix) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin and Griffin, eds.) Humana Press, NJ (1994); Sequence Analysis in Molecular Biology (Von Heijne, ed.) Academic Press (1987); and Sequence Analysis Primer (Gribskov and Devereux eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42 °C in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50 °C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 °C in a solution including 6×SSPE (20×SSPE=3M NaCl; 0.2M NaH2PO4; 0.02M EDTA, pH 7.4), 0.5% SDS, 30% formamide, 100 g/ml salmon sperm blocking DNA; followed by washes at 50 °C with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling components) transmit activation or inhibition signals comparable to a wild-type reference when in the activated state of the current disclosure. Functional variants and functional fragments of binding domains bind their cognate antigen or ligand at a level comparable to a wild-type reference.

In particular embodiments, a protein or peptide can be derived from or based on a another protein or peptide disclosed herein and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the protein or peptide disclosed herein. An insertion, deletion or substitution may be anywhere in the protein or peptide, including at the amino- or carboxy-terminus or both ends of this region.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 4th Edition (2012); F. M. Ausubel, et al. eds., Current Protocols in Molecular Biology, (2003); the series Methods In Enzymology (Academic Press, Inc.); Behlke, et al., Polymerase Chain Reaction: Theory and Technology (2019); Greenfield, ed. Antibodies, A Laboratory Manual, Second Edition (2014); and Capes-Davis and R. I. Freshney, eds. Freshney's Culture of Animal Cells 8th Edition (2021).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1-121. (canceled)

122. A method of delaying hematopoietic stem cell differentiation within a population of hematopoietic stem cells comprising administering a delta-like non-canonical Notch ligand 1 (DLK1) agonist to the population of hematopoietic stem cells, thereby delaying hematopoietic stem cell differentiation.

123. The method of claim 122, wherein the DLK1 agonist comprises a DLK1 protein, a fragment of a DLK1 protein, a nucleic acid sequence encoding DLK1, a nucleic acid sequence encoding a fragment of DLK1, or a nucleic acid sequence that binds SEQ ID NO: 40.

124. The method of claim 123, wherein
the DLK1 protein comprises the sequence of SEQ ID NO: 1 or a sequence having at least 98% sequence identity to SEQ ID NO: 1;
the fragment of the DLK1 protein comprises an extracellular domain of the DLK1 protein comprising the sequence of SEQ ID NO: 2 or any of SEQ ID NOs 36-39 or a sequence having at least 98 [% sequence identity to the sequence of SEQ ID NO: 2 or any of SEQ ID NOs 36-39;
the nucleic acid sequence encoding DLK1 comprises the sequence of SEQ ID NO: 35 or a sequence having at least 98% sequence identity to the sequence of SEQ ID NO: 35; and/or
the nucleic acid sequence that binds SEQ ID NO: 40 has the sequence of SEQ ID NO: 41 or SEQ ID NO: 42 or has a sequence having at least 98% sequence identity to SEQ ID NO: 41 or SEQ ID NO: 42.

125. The method of claim 122, further comprising culturing the population of hematopoietic stem cells on endothelial cells.

126. The method of claim 125, wherein the culturing comprises incubating in media containing growth factors selected from stem cell factor (SCF), FMS-like tyrosine kinase 3 ligand (FLT3L), thrombopoietin (TPO), interleukin (IL)-6, and/or IL-3.

127. The method of claim 122, wherein the population of hematopoietic stem cells are ex vivo hematopoietic stem cells.

128. The method of claim 122, wherein the population of hematopoietic stem cells are within a subject.

129. A method of shifting hematopoietic stem cells from a quiescent state to an activated state comprising contacting hematopoietic stem cells with a delta-like non-canonical Notch ligand 1 (DLK1) antagonist, thereby shifting hematopoietic stem cells from the quiescent state to the activated state.

130. The method of claim 129, wherein the DLK1 antagonist comprises an anti-DLK1 antibody comprising
a variable heavy chain comprising the sequence of SEQ ID NO: 43 or a sequence having 98% sequence identity to SEQ ID NO: 43 with no modifications within a complementarity determining region (CDR) and a variable light chain comprising the sequence of SEQ ID NO: 44 or a sequence having 98% sequence identity to SEQ ID NO: 44 with no modifications within a CDR;
a variable heavy chain comprising the sequence of SEQ ID NO: 45 or a sequence having 98% sequence identity to SEQ ID NO: 45 with no modifications within a CDR and a variable light chain comprising the sequence of SEQ ID NO: 46 or a sequence having 98% sequence identity to SEQ ID NO: 46 with no modifications within a CDR.
a variable heavy chain comprising the sequence of SEQ ID NO: 53 or a sequence having 98% sequence identity to SEQ ID NO: 53 with no modifications within a CDR and a variable light chain comprising the sequence of SEQ ID NO: 54 or a sequence having 98% sequence identity to SEQ ID NO: 54 with no modifications within a CDR.

131. The method of claim 129, wherein the DLK1 antagonist comprises an oligonucleotide comprising any of SEQ ID NOs: 11-32, or 40 or a sequence having 98% sequence identity to any one of SEQ ID NOs: 11-32, or 40.

132. The method of claim 129, wherein the DLK1 antagonist comprises siRNA SEQ ID NO: 33, SEQ ID NO: 59, or any one of SEQ ID NOs: 60-62 or a sequence having 98% sequence identity to SEQ ID NO: 33, SEQ ID NO: 59, or any one of SEQ ID NOs: 60-62.

133. The method of claim 129, wherein the hematopoietic stem cells are ex vivo.

134. The method of claim 129, wherein the hematopoietic stem cells are within a subject.

135. A method comprising
administering a formulation comprising stem cells with modulated DLK1 activity and a pharmaceutically acceptable carrier to a subject
wherein the modulated DLK1 activity is upregulated DLK1 activity and the subject has received or is receiving chemotherapy or another cytotoxic agent or
wherein the modulated DLK1 activity is downregulated DLK1 activity and the subject has clonal hematopoiesis.

136. The method of claim 135, wherein the DLK1 activity is upregulated by a DLK1 agonist comprising a DLK1 protein, a fragment of a DLK1 protein, a nucleic acid sequence encoding DLK1, a nucleic acid sequence encoding a fragment of DLK1, or a nucleic acid sequence that binds SEQ ID NO: 40.

137. The method of claim 136, wherein
the DLK1 protein comprises the sequence of SEQ ID NO: 1 or a sequence having at least 98% sequence identity to SEQ ID NO: 1;
the fragment of the DLK1 protein comprises an extracellular domain of the DLK1 protein comprising the sequence of SEQ ID NO: 2 or any of SEQ ID NOs 36-39 or a sequence having at least 98% sequence identity to the sequence of SEQ ID NO: 2 or any of SEQ ID NOs 36-39;
the nucleic acid sequence encoding DLK1 comprises the sequence of SEQ ID NO: 35 or a sequence having at least 98% sequence identity to the sequence of SEQ ID NO: 35; and/or
the nucleic acid sequence that binds SEQ ID NO: 40 has the sequence of SEQ ID NO: 41 or SEQ ID NO: 42 or has a sequence having at least 98% sequence identity to SEQ ID NO: 41 or SEQ ID NO: 42.

138. The method of claim 135, wherein the DLK1 activity is downregulated by a DLK1 antagonist.

139. The method of claim 138, wherein the DLK1 antagonist comprises an anti-DLK1 antibody comprising
a variable heavy chain comprising the sequence of SEQ ID NO: 43 or a sequence having 98% sequence identity to SEQ ID NO: 43 with no modifications within a complementarity determining region (CDR) and a variable light chain comprising the sequence of SEQ ID NO: 44 or a sequence having 98% sequence identity to SEQ ID NO: 44 with no modifications within a CDR;
a variable heavy chain comprising the sequence of SEQ ID NO: 45 or a sequence having 98% sequence identity to SEQ ID NO: 45 with no modifications within a CDR and a variable light chain comprising the sequence of SEQ ID NO: 46 or a sequence having 98% sequence identity to SEQ ID NO: 46 with no modifications within a CDR.
a variable heavy chain comprising the sequence of SEQ ID NO: 53 or a sequence having 98% sequence identity to SEQ ID NO: 53 with no modifications within a CDR and a variable light chain comprising the sequence of SEQ ID NO: 54 or a sequence having 98% sequence identity to SEQ ID NO: 54 with no modifications within a CDR.

140. The method of claim 138, wherein the DLK1 antagonist comprises an oligonucleotide comprising any of SEQ ID NOs: 11-32, or 40 or a sequence having 98% sequence identity to any one of SEQ ID NOs: 11-32, or 40.

141. The method of claim 138, wherein the DLK1 antagonist comprises siRNA SEQ ID NO: 33, SEQ ID NO: 59, or any one of SEQ ID NOs: 60-62 or a sequence having 98% sequence identity to SEQ ID NO: 33, SEQ ID NO: 59, or any one of SEQ ID NOs: 60-62.


SEQUENCE LISTING (62 sequences)

SEQ ID NO: 1 - protein, 383 aa, Homo sapiens
  source 1..383: note = DLK1
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNRTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 2 - protein, 304 aa, Homo sapiens
  source 1..304: note = ECD of DLK1
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNRTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQA

SEQ ID NO: 3-10 - skipped (8 entries)

SEQ ID NO: 11 - RNA, 23 nt, synthetic construct
  source 1..23: mol_type = other RNA
cctgcaacccccaaaatggattc

SEQ ID NO: 12 - RNA, 27 nt, synthetic construct
  source 1..27: mol_type = other RNA
cagaatccattttgggggttgcaggcc

SEQ ID NO: 13 - RNA, 23 nt, synthetic construct
  source 1..23: mol_type = other RNA
ctctcttaatgcatgatacagaa

SEQ ID NO: 14 - RNA, 27 nt, synthetic construct
  source 1..27: mol_type = other RNA
tattctgtatcatgcattaagagagag

SEQ ID NO: 15 - RNA, 23 nt, synthetic construct
  source 1..23: mol_type = other RNA
cttgtgtcaaatctggtgaacgc

SEQ ID NO: 16 - RNA, 27 nt, synthetic construct
  source 1..27: mol_type = other RNA
tagcgttcaccagatttgacacaagag

SEQ ID NO: 17 - RNA, 23 nt, synthetic construct
  source 1..23: mol_type = other RNA
ctctctcttaatgcatgatacag

SEQ ID NO: 18 - RNA, 27 nt, synthetic construct
  source 1..27: mol_type = other RNA
ttctgtatcatgcattaagagagagaa

SEQ ID NO: 19 - RNA, 23 nt, synthetic construct
  source 1..23: mol_type = other RNA
aaataagtatgttattctaaaat

SEQ ID NO: 20 - RNA, 27 nt, synthetic construct
  source 1..27: mol_type = other RNA
agattttagaataacatacttatttct

SEQ ID NO: 21 - RNA, 23 nt, synthetic construct
  source 1..23: mol_type = other RNA
ctcttaatgcatgatacagaata

SEQ ID NO: 22 - RNA, 27 nt, synthetic construct
  source 1..27: mol_type = other RNA
attattctgtatcatgcattaagagag

SEQ ID NO: 23 - RNA, 23 nt, synthetic construct
  source 1..23: mol_type = other RNA
aaaatggattctgcgaggatgac

SEQ ID NO: 24 - RNA, 27 nt, synthetic construct
  source 1..27: mol_type = other RNA
ttgtcatcctcgcagaatccattttgg

SEQ ID NO: 25 - RNA, 23 nt, synthetic construct
  source 1..23: mol_type = other RNA
ggcttctcaggcaatttctgcga

SEQ ID NO: 26 - RNA, 27 nt, synthetic construct
  source 1..27: mol_type = other RNA
tctcgcagaaattgcctgagaagccag

SEQ ID NO: 27 - RNA, 19 nt, synthetic construct
  source 1..19: mol_type = other RNA
cttaatgcatgatacagaa

SEQ ID NO: 28 - RNA, 19 nt, synthetic construct
  source 1..19: mol_type = other RNA
ttctgtatcatgcattaag

SEQ ID NO: 29 - RNA, 19 nt, synthetic construct
  source 1..19: mol_type = other RNA
caaacgcaatgcaaaaaca

SEQ ID NO: 30 - RNA, 19 nt, synthetic construct
  source 1..19: mol_type = other RNA
tgtttttgcattgcgtttg

SEQ ID NO: 31 - RNA, 19 nt, synthetic construct
  source 1..19: mol_type = other RNA
gcttacatatattgtcttt

SEQ ID NO: 32 - RNA, 19 nt, synthetic construct
  source 1..19: mol_type = other RNA
aaagacaatatatgtaagc

SEQ ID NO: 33 - DNA, 21 bp, synthetic construct
  source 1..21: mol_type = other DNA
ggacgatggcctctatgaatg

SEQ ID NO: 34 - DNA, 21 bp, synthetic construct
  source 1..21: mol_type = other DNA
ggttcgccaatgcgtaagagt

SEQ ID NO: 35 - DNA, 4657 bp, Homo sapiens
  source 1..4657: mol_type = other DNA
ggcagctccccggcagcggcggtggagagcgcagcgcgcagcccggtgcagccctggctttcccctcgctgcgcgcccgcgccccctttcgcgtccgcaaccagaagcccagtgcggcgccaggagccggacccgcgcccgcaccgctcccgggaccgcgaccccggccgcccagagatgaccgcgaccgaagccctcctgcgcgtcctcttgctcctgctggctttcggccacagcacctatggggctgaatgcttcccggcctgcaacccccaaaatggattctgcgaggatgacaatgtttgcaggtgccagcctggctggcagggtcccctttgtgaccagtgcgtgacctctcccggctgccttcacggactctgtggagaacccgggcagtgcatttgcaccgacggctgggacggggagctctgtgatagagatgttcgggcctgctcctcggccccctgtgccaacaacaggacctgcgtgagcctggacgatggcctctatgaatgctcctgtgcccccgggtactcgggaaaggactgccagaaaaaggacgggccctgtgtgatcaacggctccccctgccagcacggaggcacctgcgtggatgatgagggccgggcctcccatgcctcctgcctgtgcccccctggcttctcaggcaatttctgcgagatcgtggccaacagctgcacccccaacccatgcgagaacgacggcgtctgcactgacattgggggcgacttccgctgccggtgcccagccggcttcatcgacaagacctgcagccgcccggtgaccaactgcgccagcagcccgtgccagaacgggggcacctgcctgcagcacacccaggtgagctacgagtgtctgtgcaagcccgagttcacaggtctcacctgtgtcaagaagcgcgcgctgagcccccagcaggtcacccgtctgcccagcggctatgggctggcctaccgcctgacccctggggtgcacgagctgccggtgcagcagccggagcaccgcatcctgaaggtgtccatgaaagagctcaacaagaaaacccctctcctcaccgagggccaggccatctgcttcaccatcctgggcgtgctcaccagcctggtggtgctgggcactgtgggtatcgtcttcctcaacaagtgcgagacctgggtgtccaacctgcgctacaaccacatgctgcggaagaagaagaacctgctgcttcagtacaacagcggggaggacctggccgtcaacatcatcttccccgagaagatcgacatgaccaccttcagcaaggaggccggcgacgaggagatctaagcagcgttcccacagccccctctagattcttggagttccgcagagcttactatacgcggtctgtcctaatctttgtggtgttcgctatctcttgtgtcaaatctggtgaacgctacgcttacatatattgtctttgtgctgctgtgtgacaaacgcaatgcaaaaacaatcctctttctctctcttaatgcatgatacagaataataataagaatttcatctttaaatgagtaagagaaataagtatgttattctaaaatctaaactcaaatgaaatttcaaaaaagaccaaaaaaaaacaaggcaacagaaccagggctcagtgccgacgcccctaccctgggggtctcggccacatggtcctcgtgaaaccgttacgagtgctgtacatgaccacccactgtgcaaagagctacgattgctttcgttcgttaattctcacacaccacatccgactcgcactcacatccagctcaacatcgctacctttagatctttccgattgatttgagatttagcggagtgcagcggctgtgtgtcagtcagagagagccccaggttggctgctggggggccgggcctctgtgccagccctgccactaactcgctgtgtgatccttggcgagtccctatcccaatcctgggcccactttctcgcccctctcgagggaggggtttgaacaccgaccaccatggtcctctctggctctaaaactctgaattaggaacgagggggtatgaaccaaaacacttcctgaccccaacaattgggatctgatgaagacgaattctcatcctgggatccgaaaggttttggcctatcctggatgaacgcttgatgttgaaatttatcctaactgtgtttctcaaagtggggagcctcctggcccacttcccttttgggaaaaaatagcccctttgagtcataatgtgccatctggagttgccacttaggcagccagccctcccccacccctctttgcccacccagaaccccaacactatacctgcctctaccctatagagacatcccttgagacttgaatgtactttgagccgcaagtgcccctggcagaatctgagcagcaacacagccctcaccaaggttttcacagggattagtggaaattgtcacttgtggggcgcttactagttttgtggcaaatgtggtccaagacctggcccacacagccatgggtttacatttgtgttgctgagttaataatcctcagatctcaaaagacctagccagcttctgaattttgaatttgactttttttttttttttaacaaagagcatctatcttttttgcaaaaatagaaagaaatgggacagagctctgtttagcttttcagcttttggggttggacagctaaacagcccttgggtaccagataggaatattttgcttcttagattatttggataagaatctatactcttagctttattgagcattgcctgccgtgtttaccttgctccaggtgtttatttctaagtcatcatcaccttcgtcatctacagtcagtcacatcagtcacccacacacaactcacagtcacccatccattggtcacctgcgccatcgtcatgacacaatatcgacctcatcacctcatcgtcctccagccactacccaagccccctcatcctgactcaaagcccgaccagcatctgcccaaactcccaacaccccactctccccaccaagccattggtctcctcactcccggtcctttgtcatcctgattccaagccccgccccaccctcaccgcctcctccgcatcatctgcactctgcagagcccctcctacccgcctctgtccccaccaggcccctccctaccgtcagccctgagtcccacagcacgcgtccccagcccctcccagcctcaccccaaaaaccctgtcaccccaccgaccaccatcatcactggcaagccccttccccttctccagctcccgtccccactacccctcccgtcccccaatccttcccctcctgttagcccacccctcctttcctatcacccccacctttcccttcccattctccccacccttcccctccgatcccaccacccttcccttccgatccccgtcatccttcccctctgatcccccacccttcccctcctgtcctccccagccctcccttcctatcaccctcacccttcccctcccgttctccccacccttcccctcctgtctccaccagccctcccttcctatcacccccacccttctcctcccttccctcaccccaaacccctgggtcctccaccccaatctctgacaaccgccccaagtccctcttccttccccacgaagtcattcataagtcaccatctcatctcattttgagtttgttttaagaaataaaaaaattaaaaagacaaaaaaaagaattgttggcatgtgtcaggcgggcgtctggtaccctgcctggcctctctcagcgtgagacctggactgagcctgctaacgattctgttcctcagtttccccaaatagaaaattctaagattggtcagatgatcaaattatctatgttttcgatcttgatgctatagagaacagccatgttcccctgataacaagaaaaagacgtcccacggtcgaggtatccttgaccaactccccatccattcatttgttcaccgtccacaacccatctgatcaggaagcatttccaggcacctgcggagcactaggcagtgtgcggaagctgcagggcaggcaggacgagggggcagtctggaggggggtggacacacagctgtccccagggagcccagcgcggtgccggtcacatgccgtgggagtggagggtcttgagcagacgcctcccttggagaagagccaccatctcaccttcccacgcaacaaattatctattaccagcacccgatgaggccgtccctcccaaattccacagtgcccaccactcgcactaccccccccgtgccacagaagcagtgtcagcagcctcgccacggtcttcacgtcatgcagaaatggtcattccatgttctatggccaccagattctgctgagagtgtcgggatccttgactgcccagccacaccttgtgtgtgtctggtgtagtgtgtagtttacgaaacaaacggaaaagctcaaagacagcgatccaagacatgatcagcaatttggtcccaaattaggagaggaggaagaggagtatcaattttgtcaccaaaatgttaaaaaaaatgtccttggcgctctttttagctttcccgtttactaccagtccagacatgcaaaaaaaccttgtttattgaaaaataaacaacaagggctaga

SEQ ID NO: 36 - protein, 310 aa, Homo sapiens
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNGTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 37 - protein, 315 aa, Homo sapiens [sequence omitted]

SEQ ID NO: 38 - protein, 212 aa, Homo sapiens
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNGTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSSRSTAS

SEQ ID NO: 39 - protein, 203 aa, Homo sapiens [sequence omitted]

SEQ ID NO: 40 - DNA, 1347 bp, synthetic construct
  source 1..1347: mol_type = other DNA
gatgaaattcttattattattctgtatcatgcattaagagagagaaagaggattgtttttgcattgcgtttgtcacacagcagcacaaagacaatatatgtaagcgtagcgttcaccagatttgacacaagagatagcgaacaccacaaagattaggacagaccgcgtatagtaagctctgcggaactccaagaatctagagggggctgtgggaacgctgcttagatctcctcgtcgccggcctccttgctgaaggtggtcatgtcgatcttctcggggaagatgatgttgacggccaggtcctccccgctgttgtactgaagcagcaggttcttcttcttccgcagcatgtggttgtagcgcaggttggacacccaggtctcgcacttgttgaggaagacgatacccacagtgcccagcaccaccaggctggtgagcacgcccaggatggtgaagcagatggcctggccctcggtgaggagaggggttttcttgttgagctctttcatggacaccttcaggatgcggtgctccggctgctgcaccggcagctcgtgcaccccaggggtcaggcggtaggccagcccatagccgctgggcagacgggtgacctgctgggggctcagcgcgcgcttcttgacacaggtgagacctgtgaactcgggcttgcacagacactcgtagctcacctgggtgtgctgcaggcaggtgcccccgttctggcacgggctgctggcgcagttggtcaccgggcggctgcaggtcttgtcgatgaagccggctgggcaccggcagcggaagtcgcccccaatgtcagtgcagacgccgtcgttctcgcatgggttgggggtgcagctgttggccacgatctcgcagaaattgcctgagaagccaggggggcacaggcaggaggcatgggaggcccggccctcatcatccacgcaggtgcctccgtgctggcccgttgatcacacagggcccgtcctttttctggcagtcctttcccgagtacccgggggcacaggagcattcatagaggccatcgtccaggctcacgcaggtcctgttgttggcacagggggccgaggagcaggcccgaacatctctatcacagagctccccgtcccagccgtcggtgcaaatgcactgcccgggttctccacagagtccgtgaaggcagccgggagaggtcacgcactggtcacaaaggggaccctgccagccaggctggcacctgcaaacattgtcatcctcgcagaatccattttgggggttgcaggccgggaagcattcagccccataggtgctgtggccgaaagccagcaggagcaagaggacgcgcaggag

SEQ ID NO: 41 - RNA, 27 nt, synthetic construct
  source 1..27: mol_type = other RNA
gcctccttgctgaaggtggtcatgtcg

SEQ ID NO: 42 - RNA, 27 nt, synthetic construct
  source 1..27: mol_type = other RNA
ctgtgggaacgctgcttagatctcctc

SEQ ID NO: 43 - protein, 118 aa, synthetic construct
EVQLVESGGGLVQPGGSLRLSCAASGFSISDYYMAWVRQAPGKGLEWVANINYDGTNTYYADSVKGRFTISRDNSKNTLYLQMNSLRAEDTAVYYCVRSYYYYGMEYWGQGTTVTVSS

SEQ ID NO: 44 - protein, 107 aa, synthetic construct
DIQMTQSPSSLSASVGDRVTITCRASHDVSTAVAWYQQKPGKAPKLLIYSASYRYTGVPSRFSGSGSGTDFTLTISSLQPEDFATYYCQQHYRIPLTFGQGTKLEIK

SEQ ID NO: 45 - protein, 118 aa, synthetic construct
QVQLQESGPGLVKPSETLSLTCTVSGFSLSIYSVHWVRQPPGKGLEWIGLIWGGGSTDYNPSLKSRVTISKDTSKNQVSLKLSSVTAADTAVYYCARKEGNYLWFAYWGQGTLVTVSS

SEQ ID NO: 46 - protein, 113 aa, synthetic construct
DIVMTQSPDSLAVSLGERVTMNCKSSQSLLQSSNQKNYLAWYQQKPGQPPKLLVYFASTRESGVPDRFSGSGSGTDFTLTISSVQAEDVAVYYCQQHYSIPLTFGQGTKLEIK

SEQ ID NO: 47 - protein, 8 aa, synthetic construct
GFSLSIYS

SEQ ID NO: 48 - protein, 7 aa, synthetic construct
IWGGGST

SEQ ID NO: 49 - protein, 12 aa, synthetic construct
ARKEGNYLWFAY

SEQ ID NO: 50 - protein, 12 aa, synthetic construct
QSLLQSSNQKNY

SEQ ID NO: 51 - protein, 9 aa, synthetic construct
QQHYSIPLT

SEQ ID NO: 52 - protein, 112 aa, synthetic construct
QVQLQESGPGLVKPSETLSLTCTVSGFSLTSYGVSWVRQPPGKGLEWIGVIWGDGSTSYNPSLKSRVTISKDTSKNQVSLKLSSVTAADTAVYYCAKPDGPLGQGTLVTVSS

SEQ ID NO: 53 - protein, 112 aa, synthetic construct
DIVMTQSPLSLPVTPGEPASISCRSSQSLVHINGNTYLHWYLQKPGQSPQLLIYKVSNRFSGVPDRFSGSGSGTDFTLKISRVEAEDVGVYYCSQTTHVPWTFGQGTKLEIK

SEQ ID NO: 54 - protein, 8 aa, synthetic construct
GFSISDYY

SEQ ID NO: 55 - protein, 8 aa, synthetic construct
INYDGTNT

SEQ ID NO: 56 - protein, 11 aa, synthetic construct
VRSYYYYGMEY

SEQ ID NO: 57 - protein, 6 aa, synthetic construct
HDVSTA

SEQ ID NO: 58 - protein, 9 aa, synthetic construct
QQHYRIPLT

SEQ ID NO: 59 - RNA, 19 nt, synthetic construct
  source 1..19: mol_type = other RNA
agatcgtagccgcaaccaa

SEQ ID NO: 60 - RNA, 21 nt, synthetic construct
  source 1..21: mol_type = other RNA
ttggttgcggctacgatctca

SEQ ID NO: 61 - RNA, 19 nt, synthetic construct
  source 1..19: mol_type = other RNA
tcctgaaggtgtccatgaa

SEQ ID NO: 62 - RNA, 19 nt, synthetic construct
  source 1..19: mol_type = other RNA
ttcatggacaccttcagga